(19) United States
(12) Patent Application Publication
(10) Pub. No.: US 2025/0303142 A1
(43) Pub. Date: Oct. 2, 2025

(54) Electrode Assembly With Fluoroelastomer

(71) Applicant: Novocure GmbH, Baar (CH)

(72) Inventors: David Shapiro, Haifa (IL); Stas Obuchovsky, Haifa (IL); Yoram Wasserman, Haifa (IL)

(21) Appl. No.: 19/094,487

(22) Filed: Mar. 28, 2025

Related U.S. Application Data
(60) Provisional application No. 63/571,998, filed on Mar. 29, 2024.
(60) Provisional application No. 63/701,322, filed on Sep. 30, 2024.

(51) Int. Cl. A61N 1/04

(52) U.S. Cl. CPC A61N 1/0496; A61N 1/0408

(57) ABSTRACT

In one aspect, an apparatus comprises an electrode subassembly. The electrode assembly comprises at least one electrode element having a skin-facing side and a skin-facing surface and a dielectric layer on the skin-facing side of the at least one electrode element. The dielectric layer has a dielectric constant of at least 10 and comprises at least one polymer. The electrode subassembly comprises a skin-facing surface. The dielectric layer provides the skin-facing surface of the electrode subassembly. A skin contact subassembly is coupled to the electrode subassembly. The skin contact subassembly comprises a skin contact conductive adhesive or gel configured to contact skin of a subject. The skin contact conductive adhesive or gel is electrically coupled to the at least one electrode element when the skin contact subassembly is disposed against the skin-facing surface of the electrode subassembly. The skin contact subassembly is releasably coupled to the electrode subassembly.

### CROSS-REFERENCE TO RELATED APPLICATIONS

This application claims priority to and the benefit of the filing date of U.S. Provisional Patent Application Nos. 63/571,998, filed Mar. 29, 2024 and 63/701,322, filed Sep. 30, 2024, the entirety of each of which is hereby incorporated by reference herein.

### BACKGROUND

Tumor Treating Fields (TTFields) therapy is a proven approach for treating tumors using alternating electric fields at frequencies between 50 KHz-1 MHz, such as 100-500 kHz. Conventionally, the alternating electric fields are induced by electrode assemblies (e.g., arrays of capacitively coupled electrodes, also called transducer arrays) placed on opposite sides of a target location in the subject's body. When an AC voltage is applied between opposing electrode assemblies, an AC current is coupled through the electrode assemblies and into the subject's body. And higher currents are strongly correlated with higher efficacy of treatment.

The electrode assemblies used during application of TTFields typically include an electrically conductive hydrogel layer that serves as a skin contact layer that adheres to the skin of the subject. The electrically conductive hydrogel typically has a shorter lifespan than the rest of the electrode assembly. For example, with use, the skin contact layer can degrade, for example, by collecting oil and dirt, thereby reducing effectiveness of the electrically conductive hydrogel layer. The hydrogel is typically integral to the electrode assembly. Thus, upon expiration or contamination of the hydrogel, the entire electrode assembly must be disposed of and replaced.

### SUMMARY

Disclosed herein, in various aspects, are apparatuses and kits for applying TTFields.

In one aspect, an apparatus comprises an electrode subassembly. The electrode assembly comprises at least one electrode element having a skin-facing side and a skin-facing surface and a dielectric layer on the skin-facing side of the at least one electrode element. The dielectric layer has a dielectric constant of at least 10 and comprises at least one polymer. The electrode subassembly comprises a skin-facing surface. The dielectric layer provides the skin-facing surface of the electrode subassembly. A skin contact subassembly is coupled to the electrode subassembly. The skin contact subassembly comprises a skin contact conductive adhesive or gel configured to contact skin of a subject. The skin contact conductive adhesive or gel is electrically coupled to the at least one electrode element when the skin contact subassembly is disposed against the skin-facing surface of the electrode subassembly. The skin contact subassembly is releasably coupled to the electrode subassembly.

In one aspect, an apparatus comprises an electrode subassembly. The electrode subassembly includes at least one electrode element having a skin-facing side and a skin-facing surface and a dielectric layer on the skin-facing side of the at least one electrode element. The dielectric layer comprises at least one fluoroelastomer. The electrode subassembly has a skin-facing surface. The apparatus further comprises a skin contact subassembly coupled to the electrode subassembly. The skin contact subassembly comprises a skin contact conductive adhesive or gel configured to contact skin of a subject. The skin contact conductive adhesive or gel is electrically coupled to the at least one electrode element when the skin contact subassembly is disposed against the skin-facing surface of the electrode subassembly. Optionally, the skin contact subassembly is releasably coupled to the electrode subassembly

A method of using the apparatus includes removing the skin contact subassembly from the electrode subassembly.

In one aspect, a method of making an electrode subassembly is disclosed. The electrode subassembly comprises a structure having at least one electrode element, the at least one electrode element has a skin-facing side and a skin-facing surface. The method comprises applying a dielectric layer to the structure on the skin-facing side of the at least one electrode element, wherein the dielectric layer is or comprises at least one polymer (optionally, fluoroelastomer).

In one aspect, a kit comprises an electrode subassembly, the electrode subassembly comprising at least one electrode element having a skin-facing side and a skin-facing surface, and a dielectric layer on the skin-facing side of the at least one electrode element. The electrode assembly comprises a skin-facing surface. The kit further comprises a plurality of skin contact subassemblies, each skin contact subassembly configured to be removably coupled to the electrode subassembly. The skin contact subassembly comprises a skin contact conductive adhesive or gel configured to contact skin of a subject. When the skin contact subassembly is disposed against the skin-facing surface of the electrode subassembly, the skin contact conductive adhesive or gel of the skin contact subassembly is configured to electrically couple to the at least one electrode element.

Systems and methods for using the disclosed apparatuses and kits (e.g., treatment assemblies) are also disclosed.

### BRIEF DESCRIPTION OF THE DRAWINGS

FIG. 1 is a schematic top view representation of an exemplary treatment assembly as disclosed herein.

FIG. 2 is a schematic cross-sectional representations of exemplary treatment assemblies as disclosed herein, taken in the plane 2-2′ of FIG. 1.

FIG. 3A is a schematic cross-sectional representations of exemplary treatment assemblies as disclosed herein, taken in the plane 2-2′ of FIG. 1. FIG. 3B is a schematic cross-sectional representations of exemplary treatment assemblies as disclosed herein, taken in the plane 2-2′ of FIG. 1. FIG. 3C is a schematic cross-sectional representations of exemplary treatment assemblies as disclosed herein, taken in the plane 2-2′ of FIG. 1.

FIG. 4 is a schematic cross-sectional representations of exemplary treatment assemblies as disclosed herein, taken in the plane 2-2′ of FIG. 1.

FIG. 5 is a schematic cross-sectional representations of exemplary treatment assemblies as disclosed herein, taken in the plane 2-2′ of FIG. 1.

FIG. 6 is a schematic cross-sectional representations of exemplary treatment assemblies as disclosed herein, taken in the plane 2-2′ of FIG. 1.

FIG. 7 is a schematic cross-sectional representations of exemplary treatment assemblies as disclosed herein, taken in the plane 2-2′ of FIG. 1.

FIG. 8 is a schematic cross-sectional representations of exemplary treatment assemblies as disclosed herein, taken in the plane 2-2′ of FIG. 1.

FIG. 9 is a schematic cross-sectional representations of exemplary treatment assemblies as disclosed herein, taken in the plane 2-2′ of FIG. 1.

FIG. 10 is a schematic cross-sectional representations of exemplary treatment assemblies as disclosed herein, taken in the plane 2-2′ of FIG. 1.

FIG. 11 is a schematic cross-sectional representations of exemplary treatment assemblies as disclosed herein, taken in the plane 2-2′ of FIG. 1.

FIG. 12 is a schematic cross-sectional representations of exemplary treatment assemblies as disclosed herein, taken in the plane 2-2′ of FIG. 1.

FIG. 13 is a schematic cross-sectional representations of exemplary treatment assemblies as disclosed herein, taken in the plane 2-2′ of FIG. 1.

FIG. 14 is a schematic cross-sectional representations of exemplary treatment assemblies as disclosed herein, taken in the plane 2-2′ of FIG. 1.

FIG. 15 is a schematic cross-sectional representations of exemplary treatment assemblies as disclosed herein, taken in the plane 2-2′ of FIG. 1.

FIG. 16 is a schematic cross-sectional representations of exemplary treatment assemblies as disclosed herein, taken in the plane 2-2′ of FIG. 1.

FIG. 17 is a schematic cross-sectional representation of an exemplary conductive adhesive structure comprising a substrate with opposed conductive adhesive or gel layers.

FIG. 18 is a schematic cross-sectional representation of an exemplary system for aligning an electrode subassembly and a skin contact subassembly of the treatment assembly of FIG. 1.

FIG. 19 is a schematic cross-sectional representation of an exemplary system for aligning an electrode subassembly and a skin contact subassembly of the treatment assembly of FIG. 1.

FIG. 20 is a schematic cross-sectional representation of an exemplary system for aligning an electrode subassembly and a skin contact subassembly of the treatment assembly of FIG. 1.

Various embodiments are described in detail below with reference to the accompanying drawings, wherein like reference numerals represent like elements.

### DETAILED DESCRIPTION

This application describes exemplary electrode assemblies that can be used, e.g., for delivering TTFields to a subject's body and treating one or more cancers or tumors located in the subject's body.

The present invention can be understood more readily by reference to the following detailed description, examples, drawings, and claims, and their previous and following description. However, it is to be understood that this invention is not limited to the specific apparatuses, devices, systems, and/or methods disclosed unless otherwise specified, and as such, of course, can vary.

Headings are provided for convenience only and are not to be construed to limit the invention in any manner. Embodiments illustrated under any heading or in any portion of the disclosure may be combined with embodiments illustrated under the same or any other heading or other portion of the disclosure.

Any combination of the elements described herein in all possible variations thereof is encompassed by the invention unless otherwise indicated herein or otherwise clearly contradicted by context.

As used in the specification and the appended claims, the singular forms “a,” “an” and “the” include plural referents unless the context clearly dictates otherwise. Thus, it is contemplated that disclosure of a singular form of an element can provide support for embodiments in which only a single such element is provided, as well as support for embodiments in which a plurality of such elements are provided.

As used herein, the term “conductive adhesive or gel” should be understood to mean “conductive adhesive or conductive gel.” Further, the term “conductive gel” should be understood to include hydrogel.

As used herein, the term “front” refers to a skin-facing side of an element, and “rear” refers to an outwardly facing side opposite the skin-facing side of an element.

Existing electrode assemblies for providing TTFields are unitarily constructed as an assembly with one or more electrode elements and a skin contact layer. As noted above, when using existing electrode assemblies to provide TTFields, the whole construct needs to be replaced once the skin contact layer has been contaminated or has degraded. Disclosed herein are electrode assemblies that permit replacement of the skin contact layer and subsequent reuse of the electrode assembly (i.e. a 2-part array comprising an electrode subassembly and a skin contact subassembly). Because electrical current needs to be delivered through the electrode assembly and into the subject's body, one might assume that both interface layers of the 2-part array would need to be conductive. However, if a dielectric layer has a sufficient dielectric constant to capacitively couple the current into the body, and is both capable of releasing cleanly from the opposing interface layer and has sufficient structural integrity to be strong enough to survive multiple adhesion/removal cycles, then at least one interface layer need not be conductive. Disclosed herein are electrode assemblies that can further function as an interface release layer, for example, as an interface layer of an electrode subassembly for a 2-part array. In some aspects, the dielectric layer can comprise a polymer. In some aspects, the dielectric layer can comprise a fluoroelastomer. In some aspects, the dielectric layer can comprise a silicone rubber or silicone elastomer. In some aspects, the dielectric layer can comprise particles dispersed therethrough that are configured to provide an increased dielectric constant of the dielectric layer. In some aspects, the dielectric layer can comprise a polymer having particles dispersed therethrough that are configured to provide an increased dielectric constant of the dielectric layer. In some aspects, the dielectric layer can comprise a fluoroelastomer having particles dispersed therethrough that are configured to provide an increased dielectric constant of the dielectric layer. In some aspects, the dielectric layer can comprise a silicone rubber or silicone elastomer having particles dispersed therethrough that are configured to provide an increased dielectric constant of the dielectric layer.

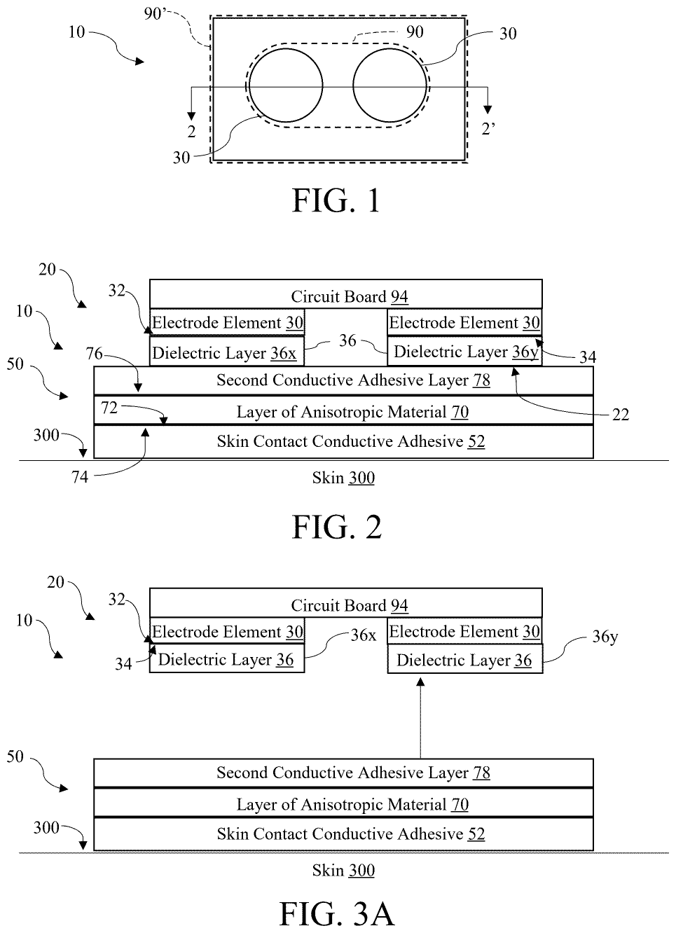

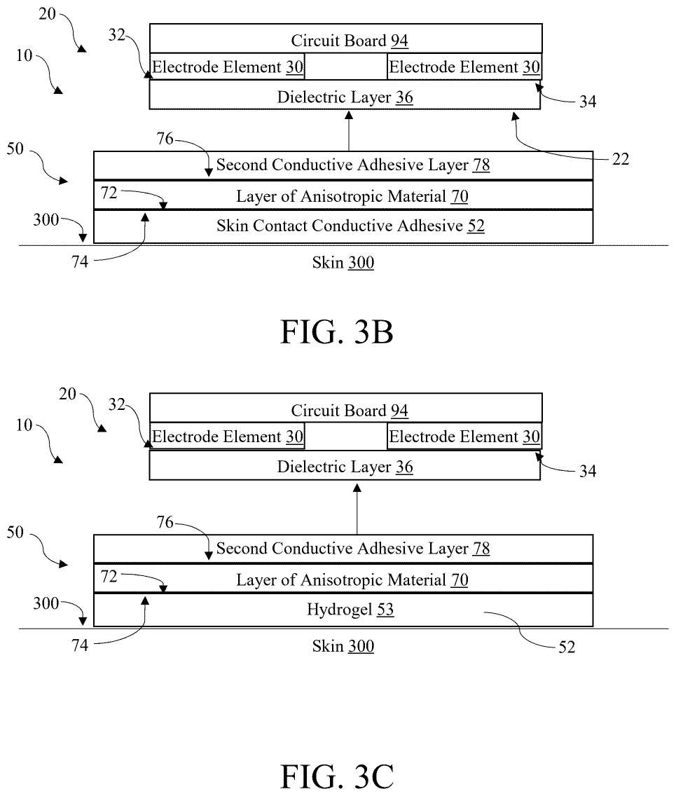

FIG. 1 illustrates a top schematic view of an apparatus 10 for providing TTFields and shows two electrode elements 30 (although the apparatus could have one, two or more than two electrode elements 30). In some optional aspects, the apparatus 10 can comprise a plurality of electrode elements 30. In these aspects, the electrode elements 30 can be wired together (e.g., using wires, or traces on a printed circuit board 94 that can optionally be a flex circuit, etc.) (FIG. 2). As discussed further herein, the apparatus 10 can have an operative conductive area. For example, as shown in FIG. 1, the operative conductive area 90 can be defined by an outer perimeter that surrounds every electrode of the at least one electrode (e.g., an areal footprint of the at least one electrode element). Also discussed herein, in alternative aspects in which the apparatus comprises a layer of anisotropic conductive material 70 (e.g., FIG. 2), the operative conductive area 90′ can be defined by the outer perimeter of the anisotropic material. Referring also to FIG. 2, showing a schematic cross-sectional representation of the apparatus 10, taken in the plane 2-2′, the apparatus 10 can comprise an electrode subassembly 20, the electrode subassembly 20 comprising at least one electrode element 30 having a skin-facing side 32 and a skin-facing surface 34. The electrode subassembly 20 can further comprise a dielectric layer 36 on the skin-facing side 32 of the at least one electrode element 30. In some aspects, the dielectric layer 36 can comprise at least one polymer, referred to herein as polymer dielectric layer 36a. In some aspects, the dielectric layer 36 can comprise at least one fluoroelastomer, referred to herein as fluoroelastomer dielectric layer 37. In some aspects, the dielectric layer 36 can comprise at least one silicone rubber or silicone elastomer with dielectric particles dispersed therein, referred to herein as silicone dielectric layer 37a. Herein, polymer dielectric layer 36a, fluoroelastomer dielectric layer 37, and silicone dielectric layer 37a are subsets of dielectric layer 36; and fluoroelastomer dielectric layer 37 and silicone dielectric layer 37a are subsets of polymer dielectric layer 36a. Herein, analogous embodiments exist for polymer dielectric layer 36a, fluoroelastomer dielectric layer 37, and silicone dielectric layer 37a when dielectric layer 36 is referred to; and, similarly, analogous embodiments exist for fluoroelastomer dielectric layer 37 and silicone dielectric layer 37a when polymer dielectric layer 36 is referred to. The electrode subassembly 20 can have a skin-facing surface 22. In some embodiments, the dielectric layer 36 is a non-adhesive polymer dielectric layer 36a. Referring to FIGS. 2 and 3A, in some aspects in which the electrode array comprises a plurality of electrode elements 30, the dielectric layer 36 can comprise a plurality of discrete elements, with each discrete element positioned over a respective electrode element 30 of the plurality of electrode elements 30. Referring to FIG. 3B and 3C, in other aspects in which the electrode array comprises multiple electrode elements 30, the dielectric layer 36 can extend across two or more (optionally, all of) the electrode elements.

The apparatus 10 can further comprise a skin contact subassembly 50 removably coupled to the electrode subassembly 20. The skin contact subassembly 50 can comprise a skin contact conductive adhesive or gel 52 configured to contact skin 300 of a subject. The skin contact conductive adhesive or gel 52 can be capacitively coupled to the at least one electrode element 30 when the skin contact subassembly 50 is disposed against the skin-facing surface of the electrode subassembly 20 (for example, when the skin contact subassembly 50 is disposed against the skin-facing surface of the dielectric layer 36 of the electrode subassembly 20 in FIG. 3). In exemplary, optional aspects, and as illustrated in FIG. 3C, the skin contact conductive adhesive or gel 52 can comprise hydrogel 53. For example, FIG. 3C illustrates a similar embodiment to that shown in FIG. 3B (and with the same labelling scheme) wherein the skin contact conductive adhesive or gel is shown as hydrogel 53. Indeed, for any layer of conductive adhesive or gel described herein, it is to be understood that the layer can be or comprise a hydrogel.

In some aspects, the skin contact subassembly 50 can be releasably coupled to the electrode subassembly 20. In other aspects, the skin contact subassembly 50 can be integrally formed with the electrode subassembly 20 so that the skin contact subassembly is non-releasably coupled to the electrode subassembly. Although the electrode subassembly 20 and the skin contact subassembly 50 are shown coupled in FIG. 2 (for example, shown as a 1-part array), this same construct could also be configured to be releasably coupled and could be represented as two separate subassemblies as shown in FIG. 3A (for example, shown as a 2-part array).

In some aspects, the dielectric layer 36 can be a single, continuous layer. For example, as shown in FIG. 3B and 3C, the single, continuous layer can extend across all of, or a plurality of, the electrode element(s) 30. In other aspects, and with reference to FIGS. 2 and 15, the dielectric layer 36 can comprise a plurality of discontinuous portions 36x, 36y. In some aspects, each discontinuous portion can be disposed on a respective skin-facing surface 34 of each electrode element 30 of the at least one electrode element.

The electrode subassembly 20 can comprise a polymer dielectric layer 36a on the skin-facing side 32 of the at least one electrode element 30, the polymer dielectric layer 36a comprising at least one polymer. In some aspects, the polymer may be a fluoroelastomer. In some aspects, the polymer may be a silicone rubber or silicone elastomer.

In some aspects, the at least one polymer of the polymer dielectric layer 36a can comprise at least one fluoroelastomer. In some aspects, the at least one fluoroelastomer of the polymer dielectric layer can be or can comprise one or more FKM (Fluorine Kautschuk Material) polymers, which, as known in the art, have a high concentration of fluorine and comprise polymerized units of vinylidene fluoride monomers (VDF) (CH₂═CF₂) and other carbon-based monomers. In exemplary aspects, such FKM polymers may be defined by ASTM International Standard D1418 and/or ISO standard 1629. In some aspects, the at least one fluoroelastomer of the polymer dielectric layer can be or can comprise poly(hexafluoropropylene-vinylidene fluoride) (i.e., p(HFP-VDF)) (e.g., optionally, VITON® fluoroelastomer, available from Chemours Company, Wilmington, DE, USA). In some aspects, the one or more FKM polymers can be or comprise Type-1 FKM polymers. In some aspects, the Type-1 FKM polymer is a copolymer (dipolymer) of vinylidene fluoride (VDF) and hexafluoropropylene (HFP). In some aspects, the one or more FKM polymers can be or comprise Type-2 FKM polymers. In some aspects, the Type-2 FKM polymer is a terpolymer of vinylidene fluoride (VDF), hexafluoropropylene (HFP), and tetrafluoroethylene (TFE). In some aspects, the one or more FKM polymers can be or comprise Type-3 FKM polymers. In some aspects, the Type-3 FKM polymer is a terpolymer of vinylidene fluoride (VDF), tetrafluoroethylene (TFE), and perfluoromethylvinylether (PMVE). In some aspects, the one or more FKM polymers can be or comprise Type-4 FKM polymers. In some aspects, the Type-4 FKM polymer is a terpolymer of vinylidene fluoride (VDF), tetrafluoroethylene (TFE), and propylene. In some aspects, the one or more FKM polymers can be or comprise Type-5 FKM polymers. In some aspects, the Type-5 FKM polymer is composed of vinylidene fluoride (VDF), hexafluoropropylene (HFP), tetrafluoroethylene (TFE), perfluoromethylvinylether (PMVE), and ethylene.

In additional aspects, the at least one polymer of the polymer dielectric layer 36a can comprise a silicone polymer, such as a silicone rubber or silicone elastomer. In additional aspects, the at least one polymer of the polymer dielectric layer 36a can include a hydrocarbon (e.g., synthetic or natural) rubber or elastomer. More generally, the at least one polymer can comprise any suitable polymer.

Further, the polymer dielectric layer 36a can comprise additional material to obtain desired properties of the polymer dielectric layer. For example, the polymer dielectric layer 36a can comprise particles dispersed therethrough. The particles can be configured to provide an increased dielectric constant of the polymer dielectric layer 36a (as compared to a remainder of the polymer dielectric layer if the particles were omitted). For example, the particles dispersed through the dielectric layer can comprise barium titanate (BaTiO₃), strontium titanate (SrTiO₃), barium strontium titanate (BaSrTiO₃), calcium copper titanate (CaCu₃Ti₄O₁₂ or CaCuTiO₃), titanium dioxide (TiO₂), or zirconium dioxide (ZrO₂). For example, the particles dispersed through the dielectric layer can comprise nanoparticles. In some optional aspects, the nanoparticles can comprise barium titanate (BaTiO₃). In some optional aspects, the nanoparticles can comprise strontium titanate (SrTiO₃). In some optional aspects, the nanoparticles can comprise barium strontium titanate (BaSrTiO₃). In some optional aspects, the nanoparticles can comprise calcium copper titanate (CaCu₃Ti₄O₁₂ or CaCuTiO₃). In some optional aspects, the nanoparticles can comprise titanium dioxide (TiO₂). In some optional aspects, the nanoparticles can comprise zirconium dioxide (ZrO₂). Further, the nanoparticles can comprise empirical formula modifications thereof (e.g., similar compounds providing similar properties, optionally, differing in quantities of respective elements, as for example, CaCu₃ Ti₄O₁₂ and CaCuTiO₃). In further aspects, the nanoparticles can comprise two or more of such compounds. Accordingly, the polymer(s) of the polymer dielectric layer 36a can have a lower dielectric constant than a desired dielectric constant for the dielectric layer, and the particles dispersed therethrough can raise the dielectric layer to the desired dielectric constant.

Examples of materials that can be used in the dielectric layer 36 include (1) Poly (VDF-HFP), (2) Poly (VDF-TrFE-CTFE) and/or Poly (VDF-TrFE-CFE), (3) one or more of barium titanate (BaTiO₃), strontium titanate (SrTiO₃), barium strontium titanate (BaSrTiO₃), calcium copper titanate (CaCu₃Ti₄O₁₂ or CaCuTiO₃), titanium dioxide (TiO₂), or zirconium dioxide (ZrO₂) particles (e.g., nanoparticles) mixed into at least one of P (VDF-HFP), Poly (VDF-TrFE-CTFE), Poly (VDF-TrFE-CFE), Poly (VDF-TrFE), PVDF, or FKM, (4) other ceramic particles (e.g., nanoparticles) mixed into at least one of P (VDF-HFP), Poly (VDF-TrFE-CTFE), Poly (VDF-TrFE-CFE), Poly (VDF-TrFE), PVDF, FKM, or other polymers, (5) one or more of barium titanate (BaTiO₃), strontium titanate (SrTiO₃), barium strontium titanate (BaSrTiO₃), calcium copper titanate (CaCu₃Ti₄O₁₂ or CaCuTiO₃), titanium dioxide (TiO₂), or zirconium dioxide (ZrO₂) particles (e.g., nanoparticles) mixed into a silicone rubber or a silicone elastomer, (5) other ceramic particles (e.g., nanoparticles) mixed into a silicone rubber or a silicone elastomer. In other embodiments, the polymer layer 30 is formed by mixing ceramic particles or nanoparticles into at least one other polymer (i.e., a polymer not listed above in this paragraph).

In some embodiments, the dielectric layer 36 can have a dielectric constant of at least 8 at at least one frequency between 50 kHz and 500 kHz. In some embodiments, the dielectric layer 36 can have a dielectric constant of at least 8 at at least one frequency between 100 kHz and 500 kHz. In some embodiments, the dielectric layer 36 can have a dielectric constant of at least 20 at at least one frequency between 50 kHz and 500 kHz. In some embodiments, the dielectric layer 36 can have a dielectric constant of at least 20 at at least one frequency between 100 kHz and 500 kHz. In some embodiments, the thickness of the polymer layer multiplied by its dielectric strength is at least 50 V, and in some embodiments the thickness of the polymer layer multiplied by its dielectric strength is at least 200 V.

In some aspects, the polymer dielectric layer 36a can have a dielectric constant of at least 8 at at least one frequency between 50 kHz and 1 MHz, such as, for example, between 100 kHz and 500 kHz. In further aspects, the polymer dielectric layer 36a can have a dielectric constant of at least 10 at at least one frequency between 50 kHz and 1 MHz, such as, for example, between 100 kHz and 500 kHz. In further aspects, the polymer dielectric layer 36a can have a dielectric constant of at least 15 at at least one frequency between 100 kHz and 500 kHz. In further aspects, the polymer dielectric layer 36a can have a dielectric constant of at least 20 at at least one frequency between 100 kHz and 500 kHz. In some aspects, the polymer dielectric layer 36a can have a dielectric constant of at least 10 at at least one frequency between 100 kHz and 500 kHz and at a temperature from 15° C. to 45° C. In further aspects, the polymer dielectric layer 36a can have a dielectric constant of at least 10 at at least one frequency between 100 kHz and 500 kHz and at a temperature from 15° C. to 45° C. In further aspects, the polymer dielectric layer 36a can have a dielectric constant of at least 15 at at least one frequency between 100 kHz and 500 kHz and at a temperature from 15° C. to 45° C. In further aspects, the polymer dielectric layer 36a can have a dielectric constant of at least 20 at at least one frequency between 100 kHz and 500 kHz and at a temperature from 15° C. to 45° C.

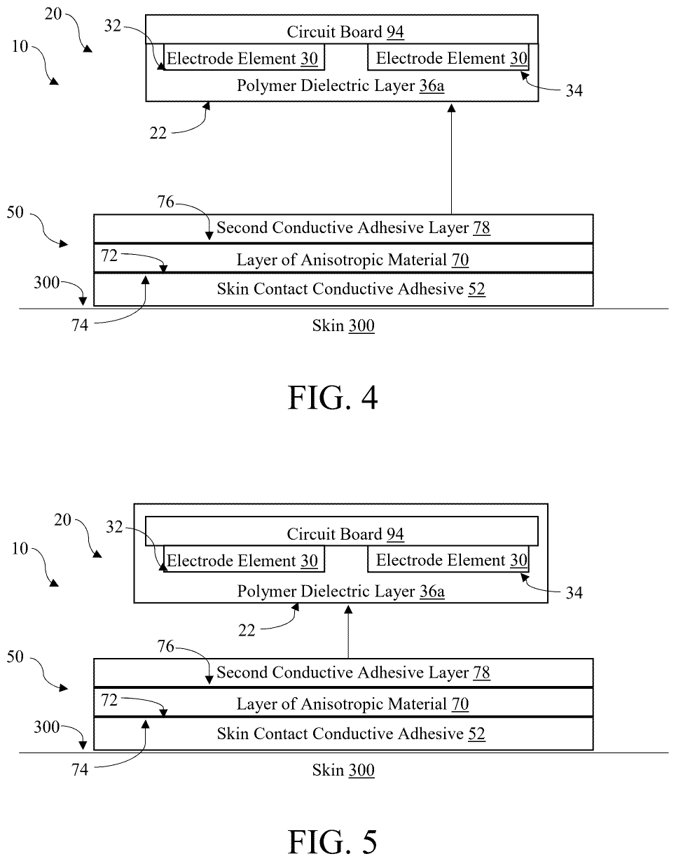

In some aspects, the electrode subassembly 20 can further comprise a circuit board 94, wherein the at least one electrode element 30 is coupled to the circuit board. The circuit board 94 can be, for example, a printed circuit board. Optionally, the circuit board 94 can be a flex circuit. Referring to FIG. 4, optionally, in these aspects, the polymer dielectric layer 36a and the circuit board 94 can cooperate to encapsulate the at least one electrode element 30. In still further aspects, and as shown in FIG. 5, the polymer dielectric layer 36a can encapsulate the circuit board 94 and the at least one electrode element 30. The polymer dielectric layer 36a can be the fluoroelastomer dielectric layer 37.

In exemplary aspects, the polymer dielectric layer 36a can have a thickness from about 1 micron to about 50 microns, or from about 2 microns to about 50 microns. In further aspects, the polymer dielectric layer 36a can have a thickness from about 2 microns to about 10 microns, or from about 2 microns to about 5 microns.

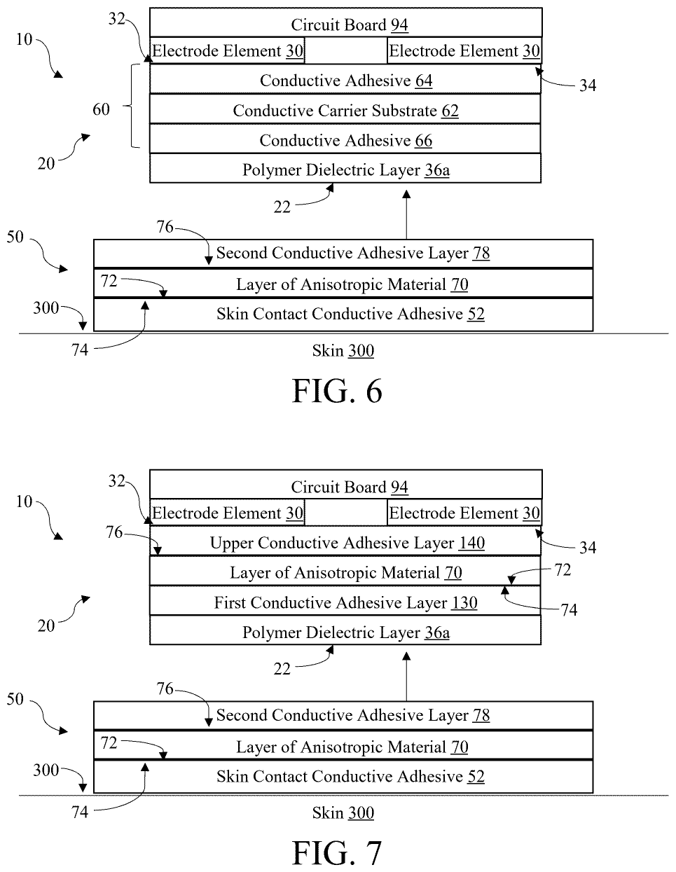

Referring to FIG. 6, in some aspects, the electrode subassembly 20 can further comprise an adhesive structure 60 positioned between the at least one electrode element 30 and the polymer dielectric layer 36a. In some aspects, the adhesive structure can comprise a double-sided tape, the double-sided tape comprising a conductive carrier substrate 62. The adhesive structure 60 can further comprise a conductive adhesive 64 between the conductive carrier substrate and the at least one electrode element 30 and a conductive adhesive 66 between the conductive carrier substrate 62 and the polymer dielectric layer 36a. In some aspects, the conductive adhesive 64 positioned between the conductive carrier substrate 62 and the at least one electrode element 30 can be a conductive acrylic adhesive. In some aspects, the conductive adhesive 66 positioned between the conductive carrier substrate and the polymer dielectric layer 36a can be a conductive silicone adhesive. In other aspects, the conductive adhesive 64 positioned between the conductive carrier substrate 62 and the at least one electrode element 30 can be a conductive silicone adhesive. In other aspects, the conductive adhesive 66 positioned between the conductive carrier substrate and the polymer dielectric layer 36a can be a conductive acrylic adhesive. In further aspects, both conductive adhesive 64 and conductive adhesive 66 can be a conductive acrylic adhesive, or both can be a conductive silicone adhesive.

Referring to FIG. 7, in some aspects, the electrode subassembly 20 can comprise a layer of anisotropic conductive material 70. In some aspects, and as shown in FIG. 7, both the electrode subassembly 20 and the skin contact subassembly 50 can comprise a layer of anisotropic conductive material 70. The anisotropic conductive material 70 is further discussed below.

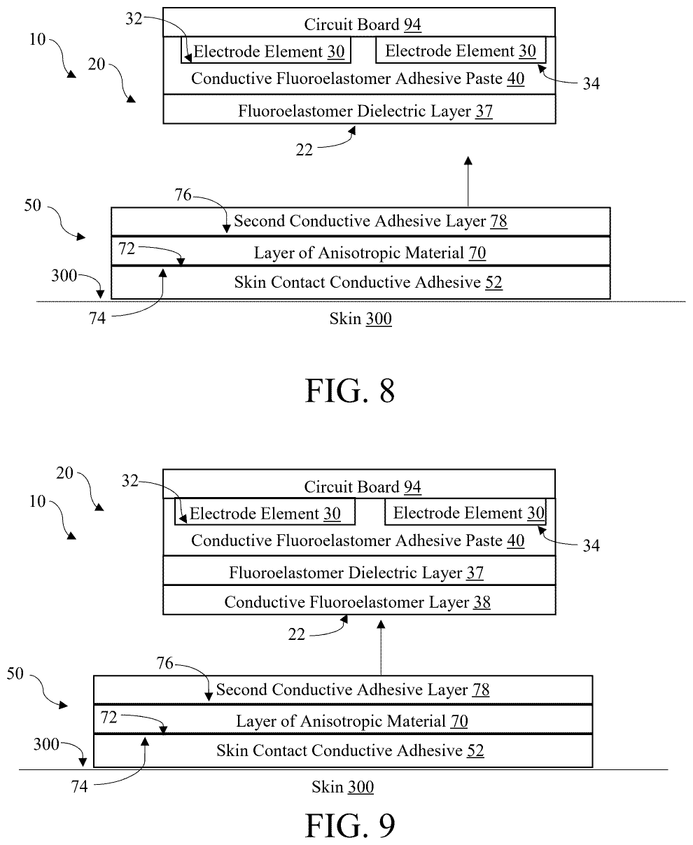

Referring to FIGS. 8-12, in some aspects, the adhesive structure 60 (FIG. 6) can comprise a conductive fluoroelastomer adhesive paste 40. For example, optionally, the conductive fluoroelastomer adhesive paste 40 can comprise poly(hexafluoropropylene-vinylidene fluoride) (p(HFP-VDF)). In further aspects, the conductive fluoroelastomer adhesive paste 40 can comprise an FKM polymer. In some aspects, the conductive fluoroelastomer adhesive paste 40 may comprise the same polymer type (in terms of polymerized monomeric units) as the polymer dielectric layer 36a (for example, the fluoroelastomer dielectric layer 37). In some aspects, the conductive fluoroelastomer adhesive paste 40 can be positioned between the at least one electrode element 30 and the polymer dielectric layer 36a (or fluoroelastomer dielectric layer 37). Alternatively, the conductive fluoroelastomer adhesive paste 40 and the circuit board 94 can cooperate to encapsulate the at least one electrode element 30, as shown in FIGS. 8-12.

Referring to FIG. 9-11, the electrode subassembly 20 can further comprise a conductive layer comprising fluoroelastomer (conductive fluoroelastomer layer 38) between the polymer dielectric layer 36a (e.g., fluoroelastomer dielectric layer 37) and the skin contact subassembly 50. In these aspects, the conductive fluoroelastomer layer 38 can define the skin-facing surface 22 of the electrode subassembly 20. Accordingly, in some aspects, the conductive fluoroelastomer layer 38 can be in contact with the skin contact subassembly 50. In further aspects, the conductive fluoroelastomer layer 38 is in contact with the polymer dielectric layer 36a (or fluoroelastomer dielectric layer 37). In other aspects, the relative positions of the polymer dielectric layer 36a and the conductive fluoroelastomer layer 38 may be reversed such that the polymer dielectric layer 36a can be in contact with the skin contact subassembly 50 and also in contact with the conductive fluoroelastomer layer 38. In some aspects, the fluoroelastomer polymer present in the conductive fluoroelastomer layer 38 is the same fluoroelastomer that is used in the fluoroelastomer dielectric layer 37, and as discussed above, these layers may be in contact with one another. In some aspects, the two layers may be fused together.

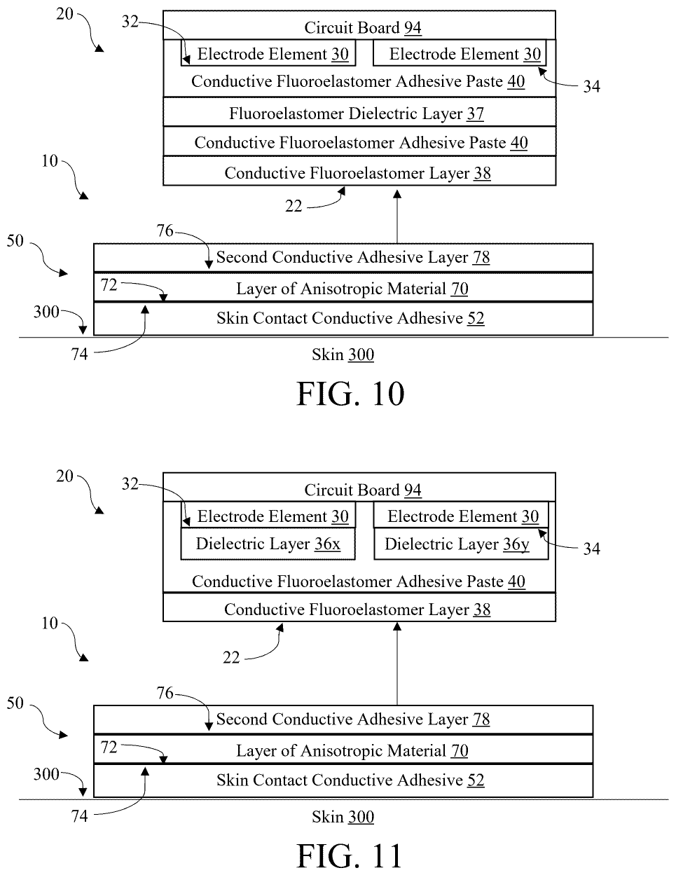

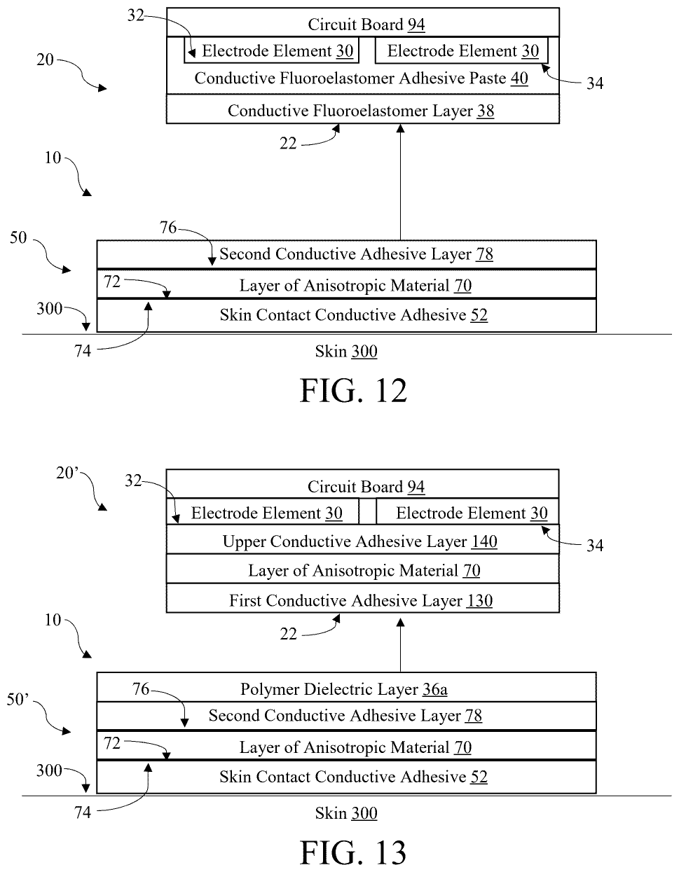

In some aspects, the conductive fluoroelastomer adhesive paste 40 can be between the polymer dielectric layer 36a (e.g., fluoroelastomer dielectric layer 37) and the conductive fluoroelastomer layer 38 (e.g., FIGS. 10-11). Referring to FIG. 10, in some aspects, the electrode subassembly 20 can comprise conductive fluoroelastomer adhesive paste 40 between the at least one electrode element 30 and the polymer dielectric layer 36a, and also between the polymer dielectric layer 36a and the conductive fluoroelastomer layer 38. In additional aspects, and with reference to FIG. 12, the polymer dielectric layer 36a can be omitted. For example, the conductive fluoroelastomer adhesive paste 40 can be disposed directly against the electrode element(s) 30.

In some aspects, there is no dielectric layer 36 (or 36a, or 37) in the electrode subassembly 20. In some aspects, there is no dielectric layer 36 (or 36a, or 37) in either the electrode subassembly 20 or the skin contact subassembly 50.

It is contemplated that the fluoroelastomer of the conductive fluoroelastomer layer 38 and/or the fluoroelastomer of the conductive fluoroelastomer adhesive paste 40 of the electrode subassembly 20 may not be conductive. Rather, the conductive fluoroelastomer layer 38 and/or the fluoroelastomer adhesive paste 40 of the electrode subassembly 20 can each comprise a respective composite material having conductive particles dispersed therethrough. For example, the conductive particles can comprise carbon. In exemplary aspects, the conductive particles can comprise one or more of: graphite powder, carbon flakes, carbon fibers, carbon granules, carbon nanotubes, carbon nanowires, carbon black powder, or carbon microcoils. In some aspects, the conductive particles can comprise metal. In exemplary aspects, the composite material can comprise polymer, for example, a fluoroelastomer, such as an FKM polymer, for example poly(hexafluoropropylene-vinylidene fluoride) (p(HFP-VDF)) with conductive particles dispersed therethrough. For brevity, these composites may be referred to herein as conductive fluoroelastomer layer 38 and conductive fluoroelastomer adhesive paste 40. As used herein, a conductive species, in some embodiments, can optionally refer to a composite material comprising the species with conductive particles dispersed therethrough.

In some aspects, the apparatus 10 does not comprise (i.e. is free of) an additional adhesive (such as a structural adhesive) between the skin contact subassembly 50 and the electrode subassembly 20. For example, the polymer dielectric layer 36a (or the conductive fluoroelastomer layer 38 of FIGS. 9-12) of the electrode subassembly 20 and the skin contact subassembly 50 can be removably coupled by a physical, non-chemical adhesion (FIGS. 3A-16). In these aspects, the polymer dielectric layer 36a (or the conductive fluoroelastomer layer 38 of FIGS. 9-12) of the electrode subassembly 20 and the skin contact subassembly 50 can be removably coupled by a Van der Waals attractive force. In some aspects, the weak Van der Waals attractive force may be supplemented. For example, the weak Van der Waals attractive force may be supplemented by an added attractive force around the perimeter, such as, for example, the positioning of one or more pairs of magnets (i.e. one of the pair on the electrode subassembly and the other of the pair on the skin contact subassembly); or a fastener, such as, for example a hook and/or loop fastener (i.e. one of the hook or loop material on the electrode subassembly and the other of the hook or loop material on the skin contact subassembly). In some aspects, the skin facing surface 22 of the electrode subassembly 20 can be smooth. In some aspects, the skin-facing surface 22 of the electrode subassembly 20 is not tacky.

In some aspects, and as shown in FIGS. 2-16, the skin contact subassembly 50 can optionally further comprise a layer of anisotropic conductive material 70 having a skin-facing side 72 with a skin-facing surface 74 and an opposing outwardly facing surface 76. The layer of anisotropic conductive material 70 can be disposed in contact with the skin contact conductive adhesive or gel 52. The at least one electrode element 30 can be in electrical contact (e.g., capacitively coupled) with the outwardly facing surface 76 of the layer of anisotropic conductive material 70 when the electrode subassembly 20 is in contact with the skin contact subassembly 50. The anisotropic conductive material 70 is further discussed below.

In some optional aspects, the skin contact conductive adhesive or gel 52 can comprise a conductive adhesive composite (described further herein). The conductive adhesive composite can comprise conductive particles dispersed therethrough. For example, the conductive particles can comprise carbon. In exemplary aspects, the conductive particles can comprise one or more of: graphite powder, carbon flakes, carbon fibers, carbon granules, carbon nanotubes, carbon nanowires, carbon black powder, or carbon microcoils. In some aspects, the conductive particles can comprise metal.

In exemplary aspects, as illustrated in FIGS. 2-16, the skin contact subassembly 50 (or 50′) can comprise a three-layer unit comprising the skin contact conductive adhesive or gel 52 (or hydrogel 53), a layer of anisotropic conductive material 70 (described further herein), and a second conductive adhesive or gel layer 78. In some aspects, the three-layer unit can further comprise an edge sealing non-conductive border such as a strip of tape, bandage or adhesive-coated foam that covers the perimeter edge of the three layers and, optionally adheres to the top and bottom surface of the three layer unit. The layer of anisotropic conductive material 70 can have a skin facing side 72 with a skin facing surface 74 and an opposing outwardly facing surface 76. The skin facing surface 74 of the layer of anisotropic conductive material 70 can be in contact with the skin contact conductive adhesive or gel 52. Optionally, the skin contact conductive adhesive or gel 52 can comprise hydrogel 53 (e.g., FIG. 3C). The outwardly facing surface 76 of the layer of anisotropic conductive material 70 can be in contact with the second conductive adhesive or gel layer 78. In still further optional aspects, the skin contact subassembly 50 can further comprise an additional skin-side conductive adhesive or gel between the layer of anisotropic conductive material 70 and the skin contact conductive adhesive or gel 52 (e.g., optionally, hydrogel 53). For example, the additional skin-side conductive adhesive or gel may be a conductive adhesive composite and the skin contact conductive adhesive or gel may be a hydrogel.

In some aspects, each of the electrode subassembly 20 and the skin contact subassembly 50 can comprise a layer of anisotropic conductive material 70 (as shown, for example, in FIG. 7).

In some aspects, the skin contact subassembly 50 can comprise acrylic adhesive. For example, in some aspects, the second conductive adhesive or gel layer 78 of the skin contact subassembly 50 in contact with the polymer dielectric layer 36a (or the conductive fluoroelastomer layer 38 of FIGS. 9 and 12) of the electrode subassembly 20 can comprise acrylic adhesive. Moreover, in some aspects, the skin contact conductive adhesive or gel 52 can comprise acrylic adhesive.

In some aspects, the electrode subassembly 20 can comprise the three-layer construct (a double layer of conductive adhesive or gel separated by a substrate layer, shown in FIG. 17) comprising the substrate layer 80, the skin-facing substrate-associated conductive adhesive or gel layer 82 and the outwardly-facing substrate-associated conductive adhesive or gel layer 84. In some aspects, the skin contact subassembly 50 can comprise the three layer construct (a double layer of conductive adhesive or gel separated by a substrate layer, shown in FIG. 17) comprising the substrate layer 80, the skin-facing substrate-associated conductive adhesive or gel layer 82 and the outwardly-facing substrate-associated conductive adhesive or gel layer 84.

Referring to FIG. 17, in some aspects, one or both of the skin contact conductive adhesive or gel 52 (or 53) and the second conductive adhesive or gel 78 of the skin contact subassembly 50 can comprise a substrate layer 80, a skin-facing substrate-associated conductive adhesive or gel layer 82, and an outwardly-facing substrate-associated conductive adhesive or gel layer 84. In some aspects, the substrate layer 80 can have a continuous, uninterrupted structure, and the substrate layer can be electrically conductive. In other aspects, the substrate layer 80 can have an at least partially open structure that is configured to permit contact of adhesive layers 82, 84 from either side of the substrate layer. In exemplary aspects, the substrate 80 can comprise a mesh or a scrim.

In some aspects, a double layer of conductive adhesive or gel separated by a substrate layer (for example, the three layer construct comprising the substrate layer 80, the skin-facing substrate-associated conductive adhesive or gel layer 82 and the outwardly-facing substrate-associated conductive adhesive or gel layer 84, shown in FIG. 17) can function as the skin contact subassembly 50 that can be removably coupled to the electrode subassembly 20. This same construct can also be used as the skin contact subassembly in kits described herein, wherein, optionally, the exposed adhesive/gel surface(s) may be protected by release liner(s). In each of these embodiments, either the skin-facing substrate-associated conductive adhesive or gel layer 82 or the outwardly-facing substrate-associated conductive adhesive or gel layer 84, or both, may be a conductive adhesive, such as a conductive adhesive composite (described herein). Further, in each of these embodiments, either the skin-facing substrate-associated conductive adhesive or gel layer 82 or the outwardly-facing substrate-associated conductive adhesive or gel layer 84, or both, may be a hydrogel.

In various aspects, one or more of the skin contact conductive adhesive or gel 52, the second conductive adhesive or gel 78, the skin-facing substrate-associated conductive adhesive or gel layer 82 or the outwardly-facing substrate-associated conductive adhesive or gel layer 84 can be hydrogel. In various aspects, one or more of the skin contact conductive adhesive or gel 52, the second conductive adhesive or gel 78, the skin-facing substrate-associated conductive adhesive or gel layer 82 or the outwardly-facing substrate-associated conductive adhesive or gel layer 84 can be a conductive adhesive composite. The conductive adhesive composite can comprise a dielectric material and conductive particles dispersed therethrough. For example, the conductive particles can comprise carbon. In exemplary aspects, the conductive particles can comprise one or more of: graphite powder, carbon flakes, carbon fibers, carbon granules, carbon nanotubes, carbon nanowires, carbon black powder, or carbon microcoils. In some aspects, the conductive particles can comprise metal. The dielectric material may be, for example, an acrylic polymer or a silicone polymer.

In exemplary aspects, the skin contact subassembly 50 can have a thickness from about 20 μm to about 400 μm, such as, for example, 100-250 μm.

In some aspects, the electrode subassembly 20 can comprise a three layer unit comprising a first conductive adhesive or gel layer 130, a layer of anisotropic conductive material 70, and an upper conductive adhesive or gel layer 140 (for example, FIGS. 7, 13, 14). The layer of anisotropic conductive material 70 can have a skin facing side 72 with a skin facing surface 74 and an opposing outwardly facing surface 76. The skin facing surface 74 of the layer of anisotropic conductive material 70 can be in contact with the first conductive adhesive or gel layer 130, and the outwardly facing surface 76 of the layer of anisotropic conductive material 70 can be in contact with the upper conductive adhesive or gel layer 140.

In some aspects either or both of the electrode subassembly 20 and the skin contact subassembly 50 may comprise a 3-layer unit (including the anisotropic material layer 70) similar to those described above.

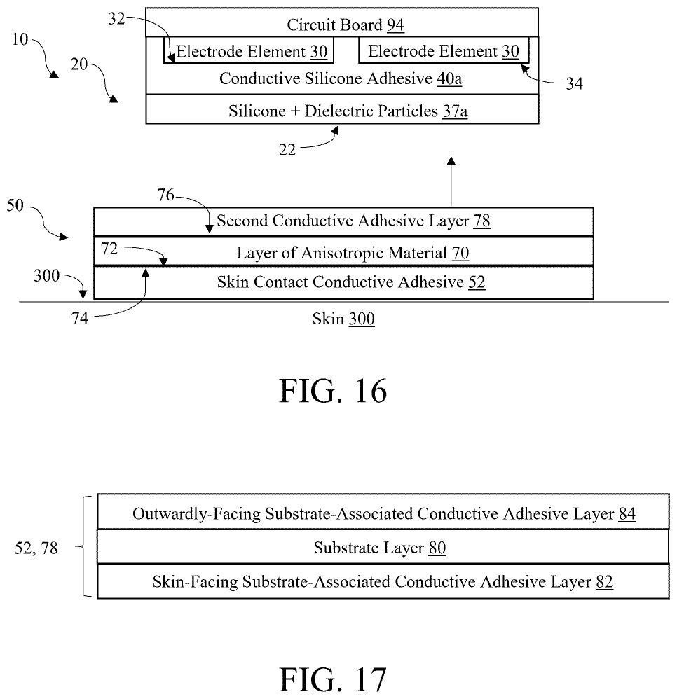

Referring to FIGS. 15 and 16, the electrode subassembly 20 can comprise a polymer dielectric layer 36a on the skin-facing side 32 of the at least one electrode element 30, wherein the polymer dielectric layer 36a comprises a silicone rubber or silicone elastomer. In some aspects, the dielectric layer comprises particles dispersed therethrough that are configured to provide an increased dielectric constant of the dielectric layer. In some aspects, the particles are nanoparticles. In some aspects, the polymer dielectric layer 36a comprises a silicone rubber or silicone elastomer comprising particles (e.g., nanoparticles) dispersed therethrough that are configured to provide an increased dielectric constant of the dielectric layer. In some aspects, the particles may be one or more of barium titanate (BaTiO₃), strontium titanate (SrTiO₃), barium strontium titanate (BaSrTiO₃), calcium copper titanate (CaCu₃Ti₄O₁₂ or CaCuTiO₃), titanium dioxide (TiO₂), or zirconium dioxide (ZrO₂) particles (e.g., nanoparticles) mixed into the silicone rubber or silicone elastomer. Herein, this dielectric layer is referred to as the silicone dielectric layer 37a. In FIGS. 15 and 16, this silicone dielectric layer is labelled “Silicone+Dielectric Particles 37a”.

Many of the same aspects described above for the apparatus 10 comprising the dielectric layer 36, the polymer dielectric layer 36a, or the fluoroelastomer dielectric layer 37 can also be constructed for the silicone dielectric layer 37a. That is, for every embodiment shown or described herein comprising the dielectric layer 36, the polymer dielectric layer 36a, or the fluoroelastomer dielectric layer 37, there also exists an analogous apparatus 10 that can also be constructed for the silicone dielectric layer 37a. For example, the silicone dielectric layer 37a can replace the dielectric layer 36 in FIG. 2 in either a unitary apparatus or (if the two subassemblies are configured to releasably couple to one another) in a 2-Part apparatus. The silicone dielectric layer 37a can be positioned over (e.g., coat) each of the one or more individual electrode elements 30, or be present as a layer that extends over the one or more electrode elements 30, or (as shown, FIG. 15), the silicone dielectric layer 37a and the circuit board 94 can cooperate to encapsulate the one or more electrode elements 30. In still further aspects, and analogous to FIG. 5, the silicone dielectric layer 37a can encapsulate the circuit board 94 and the at least one electrode element 30. Similarly, apparatuses 10 with a silicone dielectric layer 37a can be constructed to include an anisotropic material layer 70 in analogous fashion to those apparatuses 10 including the fluoroelastomer dielectric layer 37 and an anisotropic material layer 70 as described and/or illustrated elsewhere herein. As shown in FIGS. 15 and 16, the silicone dielectric layer 37a can provide the skin-facing surface of the electrode subassembly 20 and thereby act as both a dielectric layer and a release layer when the electrode subassembly 20 is releasably coupled to the skin contact subassembly 50. Other suitable polymers for the polymer dielectric layer can include other rubber polymers and sealants, including AEM (acrylic ethylene monomer) rubbers, styrene-butadiene polymers, EPDM (ethylene propylene diene monomer) polymers and the like, as known in the art. In some aspects, the dielectric layer can comprise a rubber/elastomer polymers of this type having particles dispersed therethrough that are configured to provide an increased dielectric constant of the dielectric layer.

Referring now to FIG. 16, FIG. 16 is similar to FIG. 15, but the electrode subassembly 20 additionally comprises a conductive silicone adhesive 40a between the electrode elements 30 and the silicone dielectric layer 37a. As described herein, the conductive silicone adhesive can be a silicone adhesive having conductive particles dispersed therein. The conductive silicone adhesive 40a can coat each of the one or more individual electrode elements 30, or the silicone adhesive and the circuit board 94 can cooperate to encapsulate the one or more electrode elements 30.

### Exemplary Aspects of the Disclosed Apparatuses, Kits, and Methods

As previously described above, any of the conductive adhesive or gel components of embodiments of FIGS. 2-16 may also be present as the three-layer structure of FIG. 17 and constructed similarly to that described above with respect to FIG. 17. That is, any layer of conductive adhesive or gel described herein may take the form of a double layer of conductive adhesive or gel separated by a substrate layer as described above, wherein the double layer of conductive adhesive or gel may be the same conductive adhesive or gel or a different conductive adhesive or gel either side of the substrate. For example, referring to FIGS. 2-16, in some aspects, one or both of the skin contact conductive adhesive or gel 52 or the second conductive adhesive layer 78 can comprise a substrate layer 80, a skin-facing substrate-associated conductive adhesive or gel layer 82 and an outwardly-facing substrate-associated conductive adhesive or gel layer 84. Similarly, one or both of the first conductive adhesive or gel layer 130 and the upper conductive adhesive or gel layer 140 can comprise a substrate layer 80, a skin-facing substrate-associated conductive adhesive or gel layer 82 and an outwardly-facing substrate-associated conductive adhesive or gel layer 84. In some aspects, the substrate layer 80 can have a continuous, uninterrupted structure, and the substrate layer can be electrically conductive. In other aspects, the substrate layer 80 can have an at least partially open structure that is configured to permit contact of adhesive layers 82, 84 from either side of the substrate layer. In exemplary aspects, the substrate 80 can comprise a mesh or a scrim.

In various aspects, including the embodiments of FIGS. 2-17, one or more of the second conductive adhesive or gel 78, the skin contact conductive adhesive or gel 52, the first conductive adhesive or gel layer 130, the upper conductive adhesive or gel layer 140, the skin-facing substrate-associated conductive adhesive or gel layer 82 or the outwardly-facing substrate-associated conductive adhesive or gel layer 84 can be a hydrogel. In various aspects, including the embodiments of FIGS. 2-17, one or more of the second conductive adhesive or gel 78, the skin contact conductive adhesive or gel 52, the first conductive adhesive or gel layer 130, the upper conductive adhesive or gel layer 140, the skin-facing substrate-associated conductive adhesive or gel layer 82 or the outwardly-facing substrate-associated conductive adhesive or gel layer 84 can be a conductive adhesive composite. The conductive adhesive composite can comprise a dielectric material and conductive particles dispersed therethrough. For example, the conductive particles can comprise carbon. In exemplary aspects, the conductive particles can comprise one or more of: graphite powder, carbon flakes, carbon fibers, carbon granules, carbon nanotubes, carbon nanowires, carbon black powder, or carbon microcoils. In some aspects, the conductive particles can comprise metal.

In various aspects, and as further described herein, the conductive adhesives disclosed herein can comprise a conductive adhesive composite, a hydrogel, or other suitable conductive material. For example, in some aspects, one or more of the conductive adhesives 52, 78, 130, 140, 82, 84 can be or can comprise hydrogel.

Further, as discussed above, it is contemplated that one or more of the conductive layers 52, 78, 130, 140, 82, 84 disclosed herein can comprise conductive adhesive composites (described further below). In exemplary aspects, the conductive adhesive composite can comprise a dielectric material and conductive particles dispersed within the dielectric material. In some embodiments, at least a portion of the conductive particles can define a conductive pathway through a thickness of the conductive adhesive composite. In some embodiments, it is contemplated that the conductive particles can be aligned in response to application of an electric field such that the conductive particles undergo electrophoresis. In some aspects, the conductive particles can comprise carbon. Optionally, in these aspects, the conductive particles can comprise graphite powder. Additionally, or alternatively, the conductive particles can comprise carbon flakes. Additionally, or alternatively, the conductive particles can comprise carbon granules. Additionally, or alternatively, the conductive particles can comprise carbon nanotubes. Additionally, or alternatively, the conductive particles can comprise carbon black powder. Additionally, or alternatively, the conductive particles can comprise carbon microcoils. In further aspects, the conductive adhesive composite further comprises a polar material (e.g., a polar salt). The polar salt can be a quaternary ammonium salt, such as a tetra alkyl ammonium salt. Exemplary conductive adhesive composites, as well as methods for making such conductive adhesive composites, are disclosed in U.S. Pat. No. 8,673,184 and U.S. Pat. No. 9,947,432, which are incorporated herein by reference for all purposes. In exemplary aspects, the conductive adhesive composite can be a dry carbon/salt adhesive, such as the OMNI-WAVE™ adhesive compositions manufactured and sold by FLEXCON® (Spencer, MA, USA); or ARcare® 8006 electrically conductive adhesive composition manufactured and sold by Adhesives Research, Inc. (Glen Rock, PA, USA). In further exemplary aspects, it is contemplated that the conductive adhesive composite can comprise a layer of an electrically conductive adhesive, such as for example, from use (by removal of the transfer film layer) of Electrically Conductive Adhesive Transfer Tape 9712 or Electrically Conductive Adhesive Transfer Tape 9713 (both manufactured by 3M™, St. Paul, MN, USA). As an example, the 3-layer skin contact subassembly 50 illustrated in FIG. 2 could comprise a layer of anisotropic material 70 (as described above, such as, for example, a sheet of pyrolytic graphite) sandwiched between two layers of conductive adhesive composite, which may or may not be the same. For example, the skin contact conductive adhesive or gel 52 could be an acrylic adhesive filled with carbon fibers, and the second conductive adhesive layer 78 could be an acrylic adhesive filled with carbon powder; or vice-versa. Alternatively, in some aspects, the skin contact conductive adhesive or gel 52 can be a silicone adhesive filled with carbon fibers, and the second conductive adhesive layer 78 can be a silicone adhesive filled with carbon powder; or vice-versa. Conductive silicone adhesives are available from the Dow Chemical Co. (Midland, MI, USA). Further, in some aspects, one adhesive layer may be an acrylic adhesive, and the other may be a silicone adhesive (and vice-versa); and each adhesive can have, for example, either carbon fibers or carbon powder as a conductive filler. One can further envision other options using other adhesive compositions and other conductive particle fillers. The same options for adhesive layers exist for embodiments using the 3-layer unit comprising a layer of anisotropic material 70 sandwiched between two layers of conductive adhesive composite in an electrode subassembly 20, 20. Conductive fluoroelastomer adhesive paste may be available in the Micromax® series of products available from Celanese Corp. (Irving, TX, USA) or as the carbon conductive adhesive 502 available from Delta Microscopics (Mauressac, France).

In some aspects, either the electrode subassembly 20, or the skin contact subassembly 50, or both, may comprise a layer of anisotropic conductive material 70. In some aspects, the layer of anisotropic conductive material 70 can be or can comprise a synthetic graphite. In additional aspects, the layer of anisotropic conductive material 70 can be or can comprise a layer of pyrolytic graphite, graphitized polymer film, or graphite foil made from compressed high purity exfoliated mineral graphite. In other aspects, other anisotropic materials may be suitable as the layer of anisotropic conductive material 70.

The layer of anisotropic conductive material 70 can have a first thermal conductivity in a direction that is perpendicular to the skin facing surface. In some optional aspects, thermal conductivity of the sheet in directions that are parallel to the skin facing surface can be more than two times higher than the first thermal conductivity. For example, the thermal conductivity of the sheet in directions that are parallel to the skin facing surface may be more than: 1.5 times, 2 times, 3 times, 5 times, 10 times, 20 times, 30 times, 100 times, 200 times, or even more than 1,000 times higher than the first thermal conductivity. In some embodiments, the thermal conductivity of the sheet of anisotropic material in directions that are parallel to the skin facing surface is between 1.5 times and 1000 times higher, such as between 5 times and 30 times higher, than the first thermal conductivity. The layer of anisotropic conductive material 70 can further have a first resistance in a direction that is perpendicular to the skin facing surface. In some optional aspects, resistance of the sheet in directions that are parallel to the skin facing surface can be less than half of the first resistance. For example, the resistance of the sheet 70 in directions that are parallel to the skin facing surface may be less than: 75%, 50%, 40%, 30%, 20%, 10%, 5%, 1%, 0.5%, 0.1%, or even less than 0.05% of the first resistance. In some embodiments, the resistance of the sheet of anisotropic material in directions that are parallel to the skin facing surface is between 0.05% and 75% of the first resistance, such as between 0.05% and 10% of the first resistance. In some aspects, either the electrode subassembly 20, or the skin contact subassembly 50, or both, do not include a layer of anisotropic conductive material 70.

The electrode subassembly 20 can have an operative conductive area. For example, as shown in FIG. 1, in aspects in which the electrode subassembly 20 comprises a layer of anisotropic conductive material 70, the operative conductive area 90 can be defined by the outer perimeter of the anisotropic material. In aspects in which the electrode subassembly 20 does not comprise a layer of anisotropic conductive material 70, the operative conductive area 90′ can be defined by an outer perimeter that surrounds every electrode of the at least one electrode (e.g., an areal footprint of the at least one electrode element). In some aspects, the skin contact subassembly 50 can overlie an entirety of the operative conductive area (90 or 90′). In further aspects, the skin contact subassembly 50 can extend past the operative conductive area (90 or 90′). For example, the skin contact subassembly 50 can extend past the operative conductive area (90 or 90′) by at least 1 mm, or at least 2 mm, or at least 5 mm, or at least 10 mm or more. In further aspects, the skin contact subassembly 50 can fit within the operative conductive area (90 or 90′).

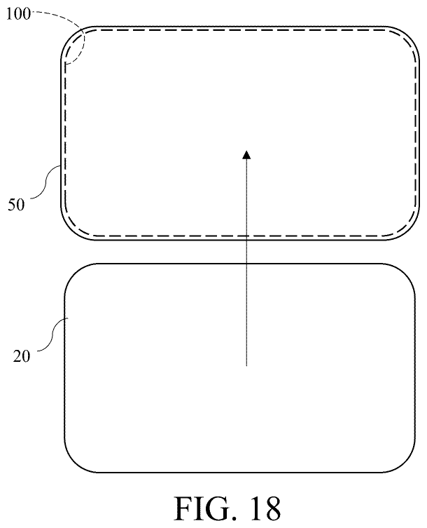

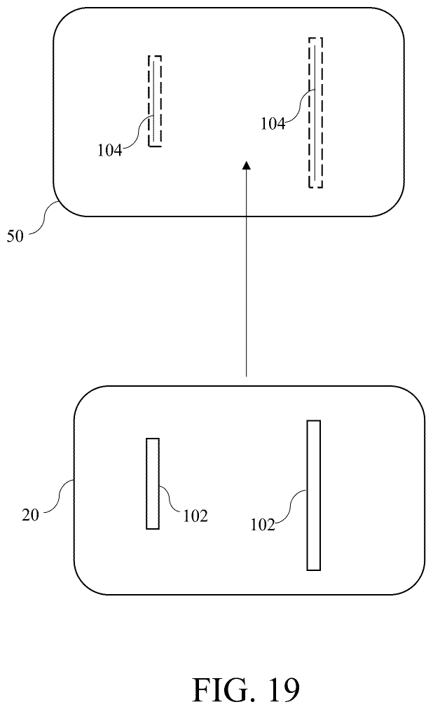

Referring to FIGS. 18-20, the skin contact subassembly 50 can comprise at least one alignment feature 100 that provides an indication of a desired position of the electrode subassembly 20 relative to the skin contact subassembly 50, in which the electrode subassembly 20 is positioned within the outer periphery of the skin contact subassembly 50 (and, conversely, the skin contact subassembly 50 overlies an entirety of the operative conductive area 90) (shown in FIG. 1). For example, referring to FIG. 18, the alignment feature 100 on the skin contact subassembly 50 can comprise an outline of at least a portion of a perimeter of the electrode subassembly 20. In other aspects, and with reference to FIG. 19, the electrode subassembly 20 can define at least one opening 102 therethrough. The alignment feature 100 can be an opening 102 and the skin contact subassembly 50 can comprise a corresponding marking 104 on the skin contact subassembly 50 that is viewable through each opening 102 of the at least one opening through the electrode subassembly 20 when the electrode subassembly is in the desired position relative to the skin contact subassembly.

In still further aspects, the skin contact subassembly 50 can have a greater footprint than the electrode subassembly 20 so that an entirety of the electrode subassembly overlies the skin contact subassembly within the footprint of the skin contact subassembly (e.g., so that some or all of the outer perimeter of the skin contact subassembly extends outwardly beyond the outer perimeter of the electrode subassembly). It is contemplated that such an arrangement can permit easier attachment and separation of the skin contact subassembly 50 and the electrode subassembly 20, permitting an outer periphery of the skin contact subassembly to be seen around the edges of the electrode subassembly. For embodiments including a layer of anisotropic conductive material in the skin contact subassembly, a larger areal footprint for the skin contact subassembly also allows for spreading of the heat and current laterally over a greater area of the skin, thereby helping to avoid the formation of any hotspots beneath the electrodes. However, in other aspects, the electrode subassembly 20 can have a greater footprint than the skin contact subassembly 50 so that an entirety of the skin contact subassembly is within the footprint of the electrode subassembly. In still other aspects, the electrode subassembly 20 and the skin contact subassembly 50 can have the same, or substantially the same footprint.

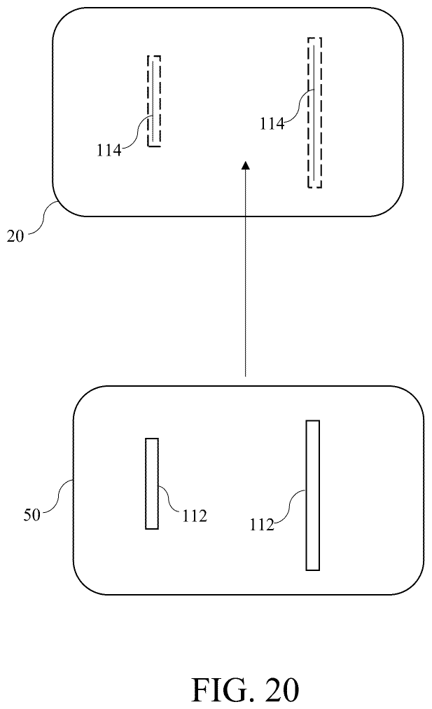

In some aspects, and with reference to FIG. 20, the skin contact subassembly 50 can define at least one opening 112 therethrough. The alignment feature 100 can comprise the at least one opening. The electrode subassembly 20 can comprise a corresponding marking 114 that is viewable through each opening 112 of the at least one opening through the skin contact subassembly 50 when the skin contact subassembly is in the desired position relative to the electrode subassembly 20.

Referring to FIGS. 1-2, in some optional aspects, the apparatus 10 can comprise a plurality of electrode elements 30. In these aspects, the electrode elements 30 can be wired together (e.g., using wires, or traces on the circuit board 94. In other aspects, the apparatus 10 (e.g., the electrode subassembly 20) can have only a single electrode element 30. Optionally, a cover tape or bandage on the electrode subassembly may be used to hold the electrodes in place.

In some aspects, and optionally, the periphery of either the electrode subassembly 20, or the skin contact subassembly 50, or both, may additionally include an affixed reversible fastener, such as, for example, a hook and loop fastener material; a fastener material based on flexible projection bodies and widened (e.g., mushroom-shaped) heads designed to interlock with similar opposingly faced fastener material of similarly-shaped (e.g., mushroom-shaped) heads; or magnets, any of which can help to support the adjoining of the electrode subassembly 20 with the skin contact subassembly 50. An example of hook and loop material is Velcro™ fastener (Velcro USA, Inc., Manchester, NH, USA), and an example of fasteners based on interlocking mushroom heads is DUAL-LOCK™ reclosable fastener (3M™, St. Paul, MN, USA).

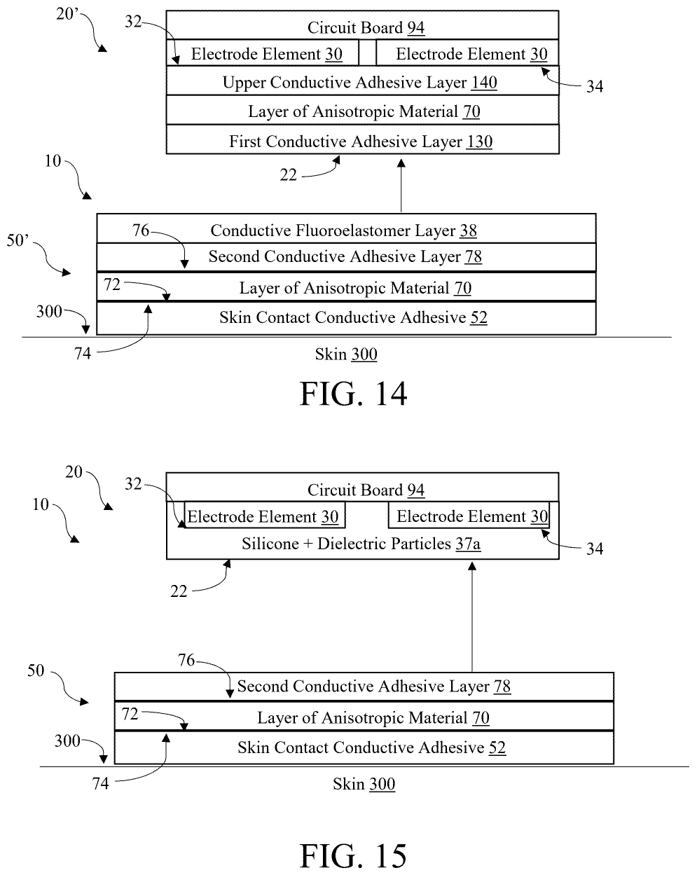

Referring to FIGS. 13-14, in some aspects, the layer comprising fluoroelastomer can be provided in a skin contact subassembly 50′. For example, an electrode subassembly 20′ can have a skin-facing surface 22. The electrode subassembly 20′ can comprise at least one electrode element 30 having a skin-facing side 32 and a skin-facing surface 34. The skin contact subassembly 50′ can be disposed against the skin-facing surface 22 of the electrode subassembly 20′. Referring to FIG. 13, in some aspects, the skin contact subassembly 50′ can comprise a polymer dielectric layer 36a that is configured to be positioned against the skin-facing surface 22 of the electrode subassembly 20′. In other aspects, and with reference to FIG. 14, the skin contact subassembly 50′ can comprise a conductive fluoroelastomer layer 38 that is configured to be positioned against the skin-facing surface 22 of the electrode subassembly 20′. In some aspects, the polymer dielectric layer 36a or conductive fluoroelastomer layer 38 can be releasably coupled to the electrode subassembly 20′.

Referring to FIGS. 13-14, in exemplary aspects, the electrode subassembly 20′ can further comprise a three layer unit comprising a first conductive adhesive or gel layer 130, a layer of anisotropic conductive material 70, and an upper conductive adhesive or gel layer 140. The layer of anisotropic conductive material 70 can have a skin facing side 72 with a skin facing surface 74 and an opposing outwardly facing surface 76. The skin facing surface 74 of the layer of anisotropic conductive material 70 can be in contact with the first conductive adhesive or gel layer 130, and the outwardly facing surface 76 of the layer of anisotropic conductive material 70 can be in contact with the upper conductive adhesive or gel layer 140. In further optional aspects, and as described herein, the skin contact subassembly 50′ can comprise a three-layer unit comprising the skin contact conductive adhesive or gel 52 (or hydrogel 53), a layer of anisotropic conductive material 70 (described further herein), and a second conductive adhesive or gel layer 78.

### Kit

A kit can comprise an electrode subassembly 20 and a plurality of skin contact subassemblies 50. Any exposed adhesive or gel surface may be protected in the kit by covering the adhesive/gel surface with a release liner. For example, in some embodiments, one or both sides of the skin contact subassembly may be provided with a release liner, each of which may be removed when the adhesive/gel surface is ready for use (for example, when the two subassemblies are to be combined, or the skin contact conductive adhesive or gel is to be adhered to the subject's skin). When the skin contact subassembly 50 is disposed against the skin-facing surface 22 of the electrode subassembly 20, the skin contact conductive adhesive or gel 52 can be configured to electrically couple to the at least one electrode element 30, and the skin contact subassembly 50 can be configured to couple to the electrode subassembly 20 as a removable unit.

In some aspects, the skin contact subassemblies 50 of the first kit can each comprise a three layer unit comprising the skin contact conductive adhesive or gel 52, a layer of anisotropic conductive material 70, and a second conductive adhesive or gel layer 78. In some aspects, the three layer unit can further comprise an edge sealing non-conductive border such as a strip of tape that covers the perimeter edge of the three layers and, optionally adheres to the top and bottom surface of the three layer unit. The layer of anisotropic conductive material 70 can have a skin facing side 72 with a skin facing surface 74 and an opposing outwardly facing surface 76. The skin facing surface 74 of the layer of anisotropic conductive material can be in contact with the skin contact conductive adhesive or gel 52. The outwardly facing surface 76 of the layer of anisotropic conductive material 70 can be in contact with the second conductive adhesive or gel layer 78. In other respects, the components of the kit (e.g., the electrode subassembly and the skin contact subassembly), the construction of the components, and the methods of use for the components of the kit can be as described above.

In exemplary aspects, a kit can be provided, the kit comprising an electrode subassembly 20′ and a plurality of skin contact subassemblies 50′. When the skin contact subassembly 50′ is disposed against the skin-facing surface 22 of the electrode subassembly 20′, the skin contact conductive adhesive or gel 52 can be configured to electrically couple to the at least one electrode element 30, and the skin contact subassembly 50′ can be configured to couple to the electrode subassembly 20′ as a removable unit.

### Method of Use

A method of using the apparatus 10 can comprise removing the skin contact subassembly 50 (or 50′) from the polymer dielectric layer 36a (or the conductive fluoroelastomer layer 38 of FIGS. 9-12) of the electrode subassembly 20 (or 20′). The polymer dielectric layer 36a can be, for example, the fluoroelastomer dielectric layer 37 or the silicone dielectric layer 37a. For example, the skin contact subassembly 50 (or 50′) that is removed can be a used, or otherwise soiled, skin contact subassembly 50 (or 50′). A new skin contact subassembly 50 (or 50′) can be positioned against the polymer dielectric layer 36a (or the conductive fluoroelastomer layer 38 of FIGS. 9-12) of the electrode subassembly 20 (or 20′) so that the new skin contact subassembly 50 (or 50′) is removably coupled to the electrode subassembly. In this way, the electrode subassembly 20 can be reused. Optionally, the polymer dielectric layer 36a (or the conductive fluoroelastomer layer 38 of FIGS. 9-12) of the electrode subassembly 20 (or 20′) can be cleaned prior to positioning the new skin contact subassembly 50 (or 50′) against the polymer dielectric layer 36a (or the conductive fluoroelastomer layer 38 of FIGS. 9-12) of the electrode subassembly 20 (or 20′).

In aspects in which the skin contact subassembly 50, 50′ (or electrode subassembly 20, 20′) comprises at least one alignment feature that provides an indication of a desired position of the electrode subassembly 20, 20′ relative to the skin contact subassembly 50, 50′, the at least one alignment feature can be used to orient the electrode subassembly 20, 20′ relative to the skin contact subassembly 50, 50′. Alignment features are discussed further herein with respect to FIGS. 18-20.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: An apparatus comprising:
an electrode subassembly comprising:
   at least one electrode element having a skin-facing side and a skin-facing surface; and
   a dielectric layer on the skin-facing side of the at least one electrode element, wherein the dielectric layer comprises at least one polymer,
   wherein the electrode subassembly comprises a skin-facing surface and wherein the dielectric layer provides the skin-facing surface of the electrode subassembly; and
a skin contact subassembly coupled to the electrode subassembly, wherein the skin contact subassembly comprises a skin contact conductive adhesive or gel configured to contact skin of a subject, wherein the skin contact conductive adhesive or gel is electrically coupled to the at least one electrode element when the skin contact subassembly is disposed against the skin-facing surface of the electrode subassembly.

Aspect 2: The apparatus of aspect 1, wherein the skin contact subassembly is releasably coupled to the electrode subassembly.

Aspect 3: The apparatus of aspect 1, wherein the skin contact subassembly is integrally formed with the electrode subassembly so that the skin contact assembly is non-releasably coupled to the electrode subassembly.

Aspect 4: The apparatus of any one of the preceding aspects, wherein the dielectric layer is a single continuous layer.

Aspect 5: The apparatus of any one of the preceding aspects, wherein the dielectric layer comprises discontinuous portions, each portion disposed on the skin-facing surface of each electrode element of the at least one electrode element.

Aspect 6: The apparatus of any one of the preceding aspects, wherein the electrode subassembly further comprises a circuit board, wherein the at least one electrode element is coupled to the circuit board, wherein the dielectric layer and the circuit board cooperate to encapsulate the at least one electrode element.

Aspect 7: The apparatus of any one of aspects 1-5, wherein the electrode subassembly further comprises a circuit board, wherein the at least one electrode element is coupled to the circuit board, wherein the dielectric layer encapsulates the circuit board and the at least one electrode element.

Aspect 8: The apparatus of aspect 6 or aspect 7, wherein the circuit board comprises a printed circuit board or flex circuit.

Aspect 9: The apparatus of any one of the preceding aspects, wherein the dielectric layer has a dielectric constant of at least 8 at at least one frequency between 50 kHz and 500 kHz.

Aspect 10: The apparatus of any one of the preceding aspects, wherein the dielectric layer has a dielectric constant of at least 10 at a frequency between 100 kHz and 500 kHz and a temperature between 15-45° C.

Aspect 11: The apparatus of any one of the preceding aspects, wherein the dielectric layer has a thickness from about 2 microns to about 50 microns.

Aspect 12: The apparatus of aspect 11, wherein the dielectric layer has a thickness from about 2 microns to about 10 microns.

Aspect 13: The apparatus of any one of the preceding aspects, wherein the electrode subassembly further comprises an adhesive structure positioned between the at least one electrode element and the dielectric layer.

Aspect 14: The apparatus of aspect 13, wherein the adhesive structure comprises:
a double-sided tape, the double-sided tape comprising a conductive carrier substrate;
a conductive adhesive between the conductive carrier substrate and the at least one electrode element; and
a conductive adhesive between the conductive carrier substrate and the dielectric layer.

Aspect 15: The apparatus of aspect 13, wherein the adhesive structure comprises:
a double-sided tape, the double-sided tape comprising a conductive carrier substrate;
a conductive acrylic adhesive between the conductive carrier substrate and the at least one electrode element; and
a conductive silicone adhesive between the conductive carrier substrate and the dielectric layer.

Aspect 16: The apparatus of any one of the preceding aspects, wherein the adhesive structure comprises a conductive fluoroelastomer adhesive paste.

Aspect 17: The apparatus of aspect 16, wherein the conductive fluoroelastomer adhesive paste comprises poly(hexafluoropropylene-vinylidene fluoride) (p(HFP-VDF)).

Aspect 18: The apparatus of any one of the preceding aspects, wherein the dielectric layer defines the skin-facing surface of the electrode subassembly.

Aspect 19: The apparatus of any one aspects 1-17, wherein the electrode subassembly further comprises a conductive layer comprising fluoroelastomer between the dielectric layer and the skin contact subassembly, wherein the conductive layer comprising fluoroelastomer defines the skin-facing surface of the electrode subassembly.

Aspect 20: The apparatus of aspect 19, wherein the conductive layer comprising fluoroelastomer is in contact with the skin contact subassembly.

Aspect 21: The apparatus of aspect 20, wherein the conductive layer comprising fluoroelastomer is in contact with the dielectric layer.

Aspect 22: The apparatus of aspect 20 or aspect 21, further comprising a conductive fluoroelastomer adhesive paste between the dielectric layer and the conductive layer comprising fluoroelastomer.

Aspect 23: The apparatus of any one of aspects 1-22, further comprising a conductive fluoroelastomer adhesive paste between the dielectric layer and the at least one electrode element.

Aspect 24: The apparatus of aspect 23, further comprising a conductive fluoroelastomer adhesive paste between the conductive layer comprising fluoroelastomer and the dielectric layer.

Aspect 25: The apparatus of any one of aspects 19-24, wherein conductive layer comprising fluoroelastomer and/or the conductive fluoroelastomer adhesive paste of the electrode subassembly each comprises a composite material having conductive particles dispersed therethrough.

Aspect 26: The apparatus of aspect 25, wherein the conductive particles comprise carbon.

Aspect 27: The apparatus of aspect 26 wherein the conductive particles comprise one or more of: graphite powder, carbon flakes, carbon fibers, carbon granules, carbon nanotubes, carbon nanowires, carbon black powder, or carbon microcoils.

Aspect 28: The apparatus of any one of aspects 25-27, wherein the conductive particles comprise metal.

Aspect 29: The apparatus of any one of the preceding aspects, wherein the apparatus does not comprise an additional adhesive between the skin contact subassembly and the electrode subassembly.

Aspect 30: The apparatus of any one of the preceding aspects, wherein the skin contact subassembly and the electrode subassembly are removably coupled by a physical, non-chemical adhesion.

Aspect 31: The apparatus of any one of the preceding aspects, wherein the skin contact subassembly and the electrode subassembly are removably coupled by a Van der Waals attractive force.

Aspect 32: The apparatus of any one of the preceding aspects, wherein the skin facing surface of the electrode subassembly is smooth, and wherein the skin contact subassembly contacts the skin facing surface of the electrode subassembly.

Aspect 33: The apparatus of any one of the preceding aspects, wherein the skin-facing surface of the electrode subassembly is not tacky.

Aspect 34: The apparatus of any one of the preceding aspects, wherein the skin contact subassembly further comprises a layer of anisotropic conductive material.

Aspect 35: The apparatus of aspect 34, wherein the a layer of anisotropic conductive material has a skin-facing side with a skin-facing surface and an opposing outwardly facing surface, wherein the layer of anisotropic conductive material is disposed in contact with the skin contact conductive adhesive or gel, and wherein the at least one electrode element is in electrical contact with the outwardly facing surface of the layer of anisotropic conductive material when the electrode subassembly is in contact with the skin contact subassembly.

Aspect 36: The apparatus of any one of the preceding aspects, wherein the skin contact conductive adhesive or gel comprises a conductive adhesive composite.

Aspect 37: The apparatus of aspect 36, wherein the conductive adhesive composite comprises conductive particles dispersed therethrough.

Aspect 38: The apparatus of aspect 37, wherein the conductive particles comprise carbon.

Aspect 39: The apparatus of aspect 38 wherein the conductive particles comprise one or more of: graphite powder, carbon flakes, carbon fibers, carbon granules, carbon nanotubes, carbon nanowires, carbon black powder, or carbon microcoils.

Aspect 40: The apparatus of aspect 37, wherein the conductive particles comprise metal.

Aspect 41: The apparatus of any one of the preceding aspects, wherein the skin contact subassembly comprises a three layer unit comprising the skin contact conductive adhesive or gel, a layer of anisotropic conductive material, and a second conductive adhesive or gel layer; wherein the layer of anisotropic conductive material has a skin facing side with a skin facing surface and an opposing outwardly facing surface; and wherein the skin facing surface of the layer of anisotropic conductive material is in contact with the skin contact conductive adhesive or gel, and the outwardly facing surface of the layer of anisotropic conductive material is in contact with the second conductive adhesive or gel layer.

Aspect 42: The apparatus of aspect 41, wherein one or both of the skin contact conductive adhesive or gel and the second conductive adhesive or gel layer comprise:

a substrate layer;

a skin-facing substrate-associated conductive adhesive or gel layer; and an outwardly-facing substrate-associated conductive adhesive or gel layer.

Aspect 43: The apparatus of aspect 42, wherein the substrate layer has a continuous, uninterrupted structure, and wherein the substrate layer is electrically conductive.

Aspect 44: The apparatus of aspect 42, wherein the substrate layer has an at least partially open structure that is configured to permit contact of adhesive layers from either side of the substrate layer.

Aspect 45: The apparatus of aspect 44, wherein the substrate comprises a mesh or a scrim.

Aspect 46: The apparatus of aspect 42, wherein one or more of the second conductive adhesive or gel, the skin-facing substrate-associated conductive adhesive or gel layer or the outwardly-facing substrate-associated conductive adhesive or gel layer is a conductive adhesive composite.

Aspect 47: The apparatus of aspect 46, wherein the conductive adhesive composite comprises conductive particles dispersed therethrough.

Aspect 48: The apparatus of aspect 47, wherein the conductive particles comprise carbon.

Aspect 49: The apparatus of aspect 48, wherein the conductive particles comprise one or more of: graphite powder, carbon flakes, carbon fibers, carbon granules, carbon nanotubes, carbon nanowires, carbon black powder, or carbon microcoils.

Aspect 50: The apparatus of any one of aspects 47-49, wherein the conductive particles comprise metal.

Aspect 51: The apparatus of any one of the preceding aspects, wherein the skin contact subassembly comprises acrylic adhesive.

Aspect 52: The apparatus of any one of aspects 34-51, wherein the layer of anisotropic conductive material is or comprises a synthetic graphite.

Aspect 53: The apparatus of any one of aspects 34-51, wherein the layer of anisotropic conductive material is or comprises a layer of pyrolytic graphite, graphitized polymer film, or graphite foil made from compressed high purity exfoliated mineral graphite.

Aspect 54: The apparatus of any one of aspects 34-53, wherein the layer of anisotropic conductive material has a first thermal conductivity in a direction that is perpendicular to the skin facing surface, and wherein thermal conductivity of the sheet in directions that are parallel to the skin facing surface is more than two times higher than the first thermal conductivity.

Aspect 55: The apparatus of any one of aspects 34-54, wherein the layer of anisotropic conductive material has a first resistance in a direction that is perpendicular to the skin facing surface, and wherein resistance of the sheet in directions that are parallel to the skin facing surface is less than half of the first resistance.

Aspect 56: The apparatus of any one of the preceding aspects, wherein the electrode subassembly has an operative conductive area, and wherein the skin contact subassembly has an areal footprint that overlies an entirety of the operative conductive area.

Aspect 57: The apparatus of any one of the preceding aspects, wherein the electrode subassembly has an operative conductive area, wherein the skin contact subassembly has an areal footprint that overlies an entirety of the operative conductive area, and wherein the skin contact subassembly comprises at least one alignment feature that provides an indication of a desired position of the electrode subassembly relative to the skin contact subassembly.

Aspect 58: The apparatus of aspect 57, wherein the alignment feature comprises an outline of at least a portion of a perimeter of the electrode subassembly.

Aspect 59: The apparatus of aspect 57, wherein the electrode subassembly defines at least one opening therethrough, wherein the alignment feature comprises a corresponding marking on the skin contact subassembly that is viewable through each opening of the at least one opening through the electrode subassembly when the electrode subassembly is in the desired position relative to the skin contact subassembly.

Aspect 60: The apparatus of aspect 57, wherein the skin contact subassembly defines at least one opening therethrough, wherein the alignment feature comprises the at least one opening, wherein the electrode subassembly comprises a corresponding marking that is viewable through each opening of the at least one opening through the skin contact subassembly when the skin contact subassembly is in the desired position relative to the electrode subassembly.

Aspect 61: The apparatus of any one of the preceding aspects, wherein the at least one polymer of the dielectric layer comprises at least one fluoroelastomer.

Aspect 62: The apparatus of aspect 61, wherein the at least one fluoroelastomer of the dielectric layer is or comprises one or more FKM polymers.

Aspect 63: The apparatus of aspect 61, wherein the at least one fluoroelastomer of the dielectric layer is or comprises at least one FKM polymer as disclosed herein.

Aspect 64: The apparatus of aspect 61, wherein the at least one fluoroelastomer of the dielectric layer is or comprises poly(hexafluoropropylene-vinylidene fluoride) (p(HFP-VDF)).

Aspect 65: The apparatus of aspect 61, wherein the at least one fluoroelastomer of the dielectric layer is or comprises at least one FKM polymer selected from (i) a copolymer of vinylidene fluoride (VDF) and hexafluoropropylene (HFP); (ii) a terpolymer of vinylidene fluoride (VDF), hexafluoropropylene (HFP), and tetrafluoroethylene (TFE); (iii) a terpolymer of vinylidene fluoride (VDF), tetrafluoroethylene (TFE), and perfluoromethylvinylether (PMVE); (iv) a terpolymer of vinylidene fluoride (VDF), tetrafluoroethylene (TFE), and propylene; or (v) FKM polymers composed of vinylidene fluoride (VDF), hexafluoropropylene (HFP), tetrafluoroethylene (TFE), perfluoromethylvinylether (PMVE), and ethylene.

Aspect 66: The apparatus of any one of aspects 1-60, wherein the at least one polymer of the dielectric layer comprises silicone.

Aspect 67: The apparatus of any one of the preceding aspects, wherein the dielectric layer comprises particles dispersed therethrough that are configured to provide an increased dielectric constant of the dielectric layer.

Aspect 68: The apparatus of any one of the preceding aspects, wherein the dielectric layer has a dielectric constant of at least 20, or at least 40, or at least 50.

Aspect 69: The apparatus of aspect 67 or 68, wherein the particles dispersed through the dielectric layer comprise nanoparticles.

Aspect 70: The apparatus of aspect 67 or 68, wherein the nanoparticles comprise BaTiO3.

Aspect 71: The apparatus of aspect 67 or 68, wherein the nanoparticles comprise BaSrTiO3.

Aspect 72: The apparatus of aspect 67 or 68, wherein the nanoparticles comprise one or more of barium titanate (BaTiO₃), strontium titanate (SrTiO₃), barium strontium titanate (BaSrTiO₃), calcium copper titanate (CaCuTiO₃ or CaCu₃Ti₄O₁₂), titanium dioxide (TiO₂), zirconium dioxide (ZrO₂), and empirical formula modifications thereof.

Aspect 73: A method of using the apparatus as in any one of the preceding aspects, the method comprising:
removing the skin contact subassembly from the electrode subassembly.

Aspect 74: The method of aspect 73, further comprising positioning a new skin contact subassembly against the skin-facing surface of the electrode subassembly so that the new skin contact subassembly is removably coupled to the electrode subassembly.

Aspect 75: The method of aspect 74, further comprising cleaning the skin-facing surface of the electrode subassembly prior to positioning the new skin contact subassembly against the skin-facing surface of the electrode subassembly.

Aspect 76: The method of aspect 74, wherein the skin contact subassembly comprises at least one alignment feature that provides an indication of a desired position of the electrode subassembly relative to the skin contact subassembly, the method comprising using the at least one alignment feature to orient the electrode subassembly relative to the skin contact subassembly, or wherein the electrode subassembly comprises at least one alignment feature that provides an indication of a desired position of the skin contact subassembly relative to the electrode subassembly, the method comprising using the at least one alignment feature to orient the skin contact subassembly relative to the electrode subassembly.

Aspect 77: A method of making an electrode subassembly, the electrode subassembly comprising a structure having at least one electrode element, the at least one electrode element having a skin-facing side and a skin-facing surface, the method comprising:
applying a dielectric layer to the structure on the skin-facing side of the at least one electrode element, wherein the dielectric layer is or comprises at least one fluoroelastomer;
optionally, wherein the electrode subassembly is a component of an apparatus of any one of Aspects 1-72, 99-108, or 111-150.

Aspect 78: The method of aspect 77, wherein applying the dielectric layer comprises melt-fusing the dielectric layer to the structure.

Aspect 79: The method of aspect 77, wherein applying the dielectric layer comprises direct printing the dielectric layer to the structure.

Aspect 80: The method of any one of aspects 77-79, wherein applying the dielectric layer comprises applying the dielectric layer to the skin-facing surface of the at least one electrode element.

Aspect 81: The method of any one of aspects 77-80, wherein applying the dielectric layer comprises adhering the dielectric layer to the structure with an adhesive structure.

Aspect 82: The method of aspect 81, wherein the adhesive structure comprises a conductive fluoroelastomer adhesive paste.

Aspect 83: The method of aspect 82, wherein the conductive fluoroelastomer adhesive paste comprises poly(hexafluoropropylene-vinylidene fluoride) (p(HFP-VDF)).

Aspect 84: The method of aspect 81, wherein the adhesive structure comprises:
a double-sided tape, the double-sided tape comprising a conductive carrier substrate;
a conductive adhesive between the conductive carrier substrate and the at least one electrode element; and
a conductive adhesive between the conductive carrier substrate and the dielectric layer.

Aspect 85: The method of aspect 81, wherein the adhesive structure comprises:
a double-sided tape, the double-sided tape comprising a conductive carrier substrate;
a conductive acrylic adhesive between the conductive carrier substrate and the at least one electrode element; and
a conductive silicone adhesive between the conductive carrier substrate and the dielectric layer.

Aspect 86: The method of any one of aspects 77-85, wherein the structure comprising the at least one electrode element comprises a circuit board and the at least one electrode element coupled thereto.

Aspect 87: The method of aspect 86, wherein the circuit board comprises a printed circuit board or flex circuit.

Aspect 88: The method of any one of aspects 82-87, wherein the at least one fluoroelastomer of the dielectric layer is or comprises one or more polymer selected from FKM polymers.

Aspect 89: The method of any one of aspects 82-88, wherein the at least one fluoroelastomer of the dielectric layer is or comprises at least one FKM polymer as disclosed herein.

Aspect 90: The method of any one of aspects 82-89, wherein the at least one fluoroelastomer of the dielectric layer is or comprises poly(hexafluoropropylene-vinylidene fluoride) (p(HFP-VDF)).

Aspect 91: A kit comprising:
an electrode subassembly comprising:
   at least one electrode element having a skin-facing side and a skin-facing surface; and
   a dielectric layer on the skin-facing side of the at least one electrode element,
   wherein the electrode assembly comprises a skin-facing surface and wherein the dielectric layer provides the skin-facing surface of the electrode subassembly; and
a plurality of skin contact subassemblies, each skin contact subassembly configured to be removably coupled to the electrode subassembly, wherein the skin contact subassembly comprises a skin contact conductive adhesive or gel configured to contact skin of a subject, wherein, when the skin contact subassembly is disposed against the skin-facing surface of the electrode subassembly, the skin contact conductive adhesive or gel of the skin contact subassembly is configured to electrically couple to the at least one electrode element.

Aspect 92: The kit of aspect 91, wherein the dielectric layer comprises a polymer.

Aspect 93: The kit of aspect 91, wherein the dielectric layer comprises a polymer comprising nanoparticles dispersed therein that are configured to provide an increased dielectric constant of the dielectric layer.

Aspect 94: The kit of aspect 91, wherein the dielectric layer comprises a fluoroelastomer.

Aspect 95: The kit of aspect 91, wherein the dielectric layer comprises a fluoroelastomer comprising nanoparticles dispersed therein that are configured to provide an increased dielectric constant of the dielectric layer.

Aspect 96: The kit of any one of aspects 91-95, wherein the dielectric layer has a dielectric constant of at least 20, or at least 40, or at least 50.

Aspect 97: The kit of any one of aspects 91-96, wherein the dielectric layer comprises a silicone rubber or silicone elastomer.

Aspect 98: The kit of any one of aspects 91-97, wherein the dielectric layer comprises a silicone rubber or silicone elastomer comprising nanoparticles dispersed therein that are configured to provide an increased dielectric constant of the dielectric layer.

Aspect 99: An apparatus comprising:
an electrode subassembly comprising:
   at least one electrode element having a skin-facing side and a skin-facing surface; and
   a dielectric layer on the skin-facing side of the at least one electrode element,
   wherein the electrode subassembly comprises a skin-facing surface; and
a skin contact subassembly coupled to the electrode subassembly, wherein the skin contact subassembly comprises a skin contact conductive adhesive or gel configured to contact skin of a subject, wherein the skin contact conductive adhesive or gel is electrically coupled to the at least one electrode element when the skin contact subassembly is disposed against the skin-facing surface of the electrode subassembly.

Aspect 100: The apparatus of aspect 99, wherein the dielectric layer provides the skin-facing surface of the electrode subassembly.

Aspect 101: The apparatus of aspect 100, wherein the dielectric layer comprises a polymer.

Aspect 102: The apparatus of aspect 100, wherein the dielectric layer comprises a polymer comprising nanoparticles dispersed therein that are configured to provide an increased dielectric constant of the dielectric layer.

Aspect 103: The apparatus of aspect 100, wherein the dielectric layer comprises a fluoroelastomer.

Aspect 104: The apparatus of aspect 100, wherein the dielectric layer comprises a fluoroelastomer comprising nanoparticles dispersed therein that are configured to provide an increased dielectric constant of the dielectric layer.

Aspect 105: The apparatus of aspect 101 or 102, wherein the dielectric layer has a dielectric constant of at least 20, or at least 40, or at least 50.

Aspect 106: The apparatus of aspect 103 or 104, wherein the dielectric layer has a dielectric constant of at least 20, or at least 40, or at least 50.

Aspect 107: The apparatus of aspect 100, wherein the dielectric layer comprises a silicone rubber or silicone elastomer.

Aspect 108: The apparatus of aspect 100, wherein the dielectric layer comprises a silicone rubber or silicone elastomer comprising nanoparticles dispersed therein that are configured to provide an increased dielectric constant of the dielectric layer.

Aspect 109: The kit of aspect 107 or 108, wherein the dielectric layer has a dielectric constant of at least 20, or at least 40, or at least 50.

Aspect 110: A kit comprising:
an electrode subassembly as described herein; and
at least one skin contact subassembly as described herein.

Aspect 111: An apparatus comprising:
an electrode subassembly comprising:
   at least one electrode element having a skin-facing side and a skin-facing surface; and
   a dielectric layer on the skin-facing side of the at least one electrode element, wherein the dielectric layer has a dielectric constant of at least 10 and comprises at least one polymer,
   wherein the electrode subassembly comprises a skin-facing surface and wherein the dielectric layer provides the skin-facing surface of the electrode subassembly; and
a skin contact subassembly coupled to the electrode subassembly, wherein the skin contact subassembly comprises a skin contact conductive adhesive or gel configured to contact skin of a subject, wherein the skin contact conductive adhesive or gel is electrically coupled to the at least one electrode element when the skin contact subassembly is disposed against the skin-facing surface of the electrode subassembly; and
wherein the skin contact subassembly is releasably coupled to the electrode subassembly.

Aspect 112: The apparatus of aspect 111, wherein the at least one polymer of the dielectric layer comprises particles dispersed therethrough that are configured to provide an increased dielectric constant of the dielectric layer.

Aspect 113: The apparatus of aspect 112, wherein the particles dispersed through the dielectric layer comprise one or more of barium titanate (BaTiO₃), strontium titanate (SrTiO₃), barium strontium titanate (BaSrTiO₃), calcium copper titanate (CaCuTiO₃ or CaCu₃Ti₄O₁₂), titanium dioxide (TiO₂), zirconium dioxide (ZrO₂), and empirical formula modifications thereof.

Aspect 114: The apparatus of aspect 112, wherein the at least one polymer of the dielectric layer is or comprises a rubber or elastomer.

Aspect 115: The apparatus of aspect 112, wherein the at least one polymer of the dielectric layer is or comprises a silicone rubber or silicone elastomer.

Aspect 116: The apparatus of any one of aspects 112-115, wherein the dielectric layer is a single continuous layer.

Aspect 117: The apparatus of any one of aspects 112-115 aspect, wherein the dielectric layer comprises discontinuous portions, each portion disposed on the skin-facing surface of each electrode element of the at least one electrode element.

Aspect 118: The apparatus of any one of aspects 112-117 aspect, wherein the electrode subassembly further comprises a circuit board, wherein the at least one electrode element is coupled to the circuit board, wherein the dielectric layer and the circuit board cooperate to encapsulate the at least one electrode element.

Aspect 119: The apparatus of any one of aspects 112-118 aspect, wherein the electrode subassembly further comprises an adhesive layer or an adhesive structure positioned between the at least one electrode element and the dielectric layer.

Aspect 120: The apparatus of aspect 119, wherein the at least one polymer of the dielectric layer is or comprises a silicone rubber or silicone elastomer, and wherein the adhesive layer or adhesive structure comprises a conductive silicone adhesive.

Aspect 121: The apparatus of aspect 112, wherein the at least one polymer of the dielectric layer is or comprises a silicone rubber or silicone elastomer, and wherein the electrode subassembly further comprises a conductive layer comprising a silicone polymer.

Aspect 122: The apparatus of any one of aspects 112-121 aspect, wherein the skin facing surface of the electrode subassembly is smooth and not tacky, and wherein the skin contact subassembly contacts the skin facing surface of the electrode subassembly.

Aspect 123: The apparatus of any one of aspects 112-122 aspect, wherein the skin contact subassembly further comprises a layer of anisotropic conductive material.

Aspect 124: The apparatus of aspect 123, wherein the layer of anisotropic conductive material has a skin-facing side with a skin-facing surface and an opposing outwardly facing surface, wherein the layer of anisotropic conductive material is disposed in contact with the skin contact conductive adhesive or gel, and wherein the at least one electrode element is in electrical contact with the outwardly facing surface of the layer of anisotropic conductive material when the electrode subassembly is in contact with the skin contact subassembly.

Aspect 125: The apparatus of aspect 123, wherein the skin contact subassembly comprises a three layer unit comprising the skin contact conductive adhesive or gel, the layer of anisotropic conductive material, and a second conductive adhesive or gel layer; wherein the layer of anisotropic conductive material has a skin facing side with a skin facing surface and an opposing outwardly facing surface; and wherein the skin facing surface of the layer of anisotropic conductive material is in contact with the skin contact conductive adhesive or gel, and the outwardly facing surface of the layer of anisotropic conductive material is in contact with the second conductive adhesive or gel layer.

Aspect 126: The apparatus of aspect 123, wherein the layer of anisotropic conductive material is or comprises graphite.

Aspect 127: The apparatus of any one of aspects 112-126 aspect, wherein the electrode subassembly further comprises a layer of anisotropic conductive material.

Aspect 128: A method of using the apparatus of any one of aspects 112-127 aspect, the method comprising:
removing the skin contact subassembly from the electrode subassembly.

Aspect 129: The method of aspect 128, further comprising positioning a new skin contact subassembly against the skin-facing surface of the electrode subassembly so that the new skin contact subassembly is removably coupled to the electrode subassembly.

Aspect 130: An apparatus comprising:
an electrode subassembly comprising:
   at least one electrode element having a skin-facing side and a skin-facing surface; and
   a dielectric layer on the skin-facing side of the at least one electrode element, wherein the dielectric layer has a dielectric constant of at least 10 and comprises a silicone rubber or silicone elastomer having particles dispersed therethrough that are configured to provide an increased dielectric constant of the dielectric layer,
   wherein the electrode subassembly comprises a skin-facing surface and wherein the dielectric layer provides the skin-facing surface of the electrode subassembly; and
a skin contact subassembly coupled to the electrode subassembly, wherein the skin contact subassembly comprises a skin contact conductive adhesive or gel configured to contact skin of a subject, wherein the skin contact conductive adhesive or gel is electrically coupled to the at least one electrode element when the skin contact subassembly is disposed against the skin-facing surface of the electrode subassembly; and
wherein the skin contact subassembly is releasably coupled to the electrode subassembly.

Aspect 131: An apparatus comprising:
an electrode subassembly comprising:
   at least one electrode element having a skin-facing side and a skin-facing surface; and
   a dielectric layer on the skin-facing side of the at least one electrode element, wherein the dielectric layer comprises at least one fluoroelastomer,
   wherein the electrode subassembly comprises a skin-facing surface and wherein the dielectric layer provides the skin-facing surface of the electrode subassembly; and
a skin contact subassembly coupled to the electrode subassembly, wherein the skin contact subassembly comprises a skin contact conductive adhesive or gel configured to contact skin of a subject, wherein the skin contact conductive adhesive or gel is electrically coupled to the at least one electrode element when the skin contact subassembly is disposed against the skin-facing surface of the electrode subassembly; and
wherein the skin contact subassembly is releasably coupled to the electrode subassembly.

Aspect 132: The apparatus of aspect 131, wherein the at least one fluoroelastomer of the dielectric layer is or comprises one or more FKM polymers. Aspect 132A: The apparatus of aspect 131, wherein the at least one fluoroelastomer of the dielectric layer is or comprises at least one FKM polymer selected from (i) a copolymer of vinylidene fluoride (VDF) and hexafluoropropylene (HFP); (ii) a terpolymer of vinylidene fluoride (VDF), hexafluoropropylene (HFP), and tetrafluoroethylene (TFE); (iii) a terpolymer of vinylidene fluoride (VDF), tetrafluoroethylene (TFE), and perfluoromethylvinylether (PMVE); (iv) a terpolymer of vinylidene fluoride (VDF), tetrafluoroethylene (TFE), and propylene; or (v) FKM polymers composed of vinylidene fluoride (VDF), hexafluoropropylene (HFP), tetrafluoroethylene (TFE), perfluoromethylvinylether (PMVE), and ethylene.

Aspect 133: The apparatus of aspect 131, wherein the at least one fluoroelastomer of the dielectric layer is or comprises poly(hexafluoropropylene-vinylidene fluoride) (p(HFP-VDF)).

Aspect 134: The apparatus of any one of aspects 131-133, wherein the dielectric layer is a single continuous layer.

Aspect 135: The apparatus of any one of aspects 131-133, wherein the dielectric layer comprises discontinuous portions, each portion disposed on the skin-facing surface of each electrode element of the at least one electrode element.

Aspect 136: The apparatus of any one of aspects 131-135, wherein the electrode subassembly further comprises a circuit board, wherein the at least one electrode element is coupled to the circuit board, wherein the dielectric layer and the circuit board cooperate to encapsulate the at least one electrode element.

Aspect 137: The apparatus of any one of aspects 131-136, wherein the dielectric layer has a dielectric constant of at least 8 at at least one frequency between 50 kHz and 500 kHz and at at least one temperature between 15-45° C.

Aspect 138: The apparatus of any one of aspects 131-137, wherein the electrode subassembly further comprises an adhesive layer or an adhesive structure positioned between the at least one electrode element and the dielectric layer.

Aspect 139: The apparatus of aspect 138, wherein the adhesive layer or adhesive structure comprises a conductive fluoroelastomer adhesive paste.

Aspect 140: The apparatus of any one of aspects 131-139, wherein the electrode subassembly further comprises a conductive layer comprising fluoroelastomer.

Aspect 141: The apparatus of any one of aspects 131-140, wherein the skin facing surface of the electrode subassembly is smooth and not tacky, and wherein the skin contact subassembly contacts the skin facing surface of the electrode subassembly.

Aspect 142: The apparatus of any one of aspects 131-140, wherein the skin contact subassembly further comprises a layer of anisotropic conductive material.

Aspect 143: The apparatus of aspect 142, wherein the layer of anisotropic conductive material has a skin-facing side with a skin-facing surface and an opposing outwardly facing surface, wherein the layer of anisotropic conductive material is disposed in contact with the skin contact conductive adhesive or gel, and wherein the at least one electrode element is in electrical contact with the outwardly facing surface of the layer of anisotropic conductive material when the electrode subassembly is in contact with the skin contact subassembly.

Aspect 144: The apparatus of aspect 142, wherein the skin contact subassembly comprises a three layer unit comprising the skin contact conductive adhesive or gel, the layer of anisotropic conductive material, and a second conductive adhesive or gel layer; wherein the layer of anisotropic conductive material has a skin facing side with a skin facing surface and an opposing outwardly facing surface; and wherein the skin facing surface of the layer of anisotropic conductive material is in contact with the skin contact conductive adhesive or gel, and the outwardly facing surface of the layer of anisotropic conductive material is in contact with the second conductive adhesive or gel layer.

Aspect 145: The apparatus of aspect 142, wherein the layer of anisotropic conductive material is or comprises graphite.

Aspect 146: The apparatus of any one of aspects 131-145, wherein the electrode subassembly further comprises a layer of anisotropic conductive material.

Aspect 147: The apparatus of any one of aspects 131-146, wherein the dielectric layer comprises particles dispersed therethrough that are configured to provide an increased dielectric constant of the dielectric layer.

Aspect 148: The apparatus of aspect 147, wherein the particles dispersed through the dielectric layer comprise one or more of barium titanate (BaTiO₃), strontium titanate (SrTiO₃), barium strontium titanate (BaSrTiO₃), calcium copper titanate (CaCuTiO₃ or CaCu₃Ti₄O₁₂), titanium dioxide (TiO₂), zirconium dioxide (ZrO₂), and empirical formula modifications thereof.

Aspect 149: A method of using the apparatus of any one of aspects 131-148, the method comprising:
removing the skin contact subassembly from the electrode subassembly.

Aspect 150: The method of aspect 149, further comprising positioning a new skin contact subassembly against the skin-facing surface of the electrode subassembly so that the new skin contact subassembly is removably coupled to the electrode subassembly.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof. For example, various combinations of layer arrangements are disclosed with reference to the illustrated embodiments. Logical combinations and omissions of the different layers are contemplated. As another example, various skin contact subassemblies are illustrated in combination with different electrode subassemblies. It should be understood that the electrode subassemblies and skin contact subassemblies can logically be interchanged within the spirit and scope of the present disclosure.

What is claimed is:

1. An apparatus comprising:
an electrode subassembly comprising:
at least one electrode element having a skin-facing side and a skin-facing surface; and
a dielectric layer on the skin-facing side of the at least one electrode element, wherein the dielectric layer has a dielectric constant of at least 10 and comprises at least one polymer,
wherein the electrode subassembly comprises a skin-facing surface and wherein the dielectric layer provides the skin-facing surface of the electrode subassembly; and
a skin contact subassembly coupled to the electrode subassembly, wherein the skin contact subassembly comprises a skin contact conductive adhesive or gel configured to contact skin of a subject, wherein the skin contact conductive adhesive or gel is electrically coupled to the at least one electrode element when the skin contact subassembly is disposed against the skin-facing surface of the electrode subassembly; and
wherein the skin contact subassembly is releasably coupled to the electrode subassembly.

2. The apparatus of claim 1, wherein the at least one polymer of the dielectric layer comprises particles dispersed therethrough that are configured to provide an increased dielectric constant of the dielectric layer.

3. The apparatus of claim 2, wherein the particles dispersed through the dielectric layer comprise one or more of barium titanate (BaTiO₃), strontium titanate (SrTiO₃), barium strontium titanate (BaSrTiO₃), calcium copper titanate (CaCuTiO₃ or CaCu₃Ti₄O₁₂), titanium dioxide (TiO₂), zirconium dioxide (ZrO₂), and empirical formula modifications thereof.

4. The apparatus of claim 2, wherein the at least one polymer of the dielectric layer is or comprises a rubber or elastomer.

5. The apparatus of claim 2, wherein the at least one polymer of the dielectric layer is or comprises a silicone rubber or a silicone elastomer.

6. The apparatus of claim 2, wherein the dielectric layer is a single continuous layer.

7. The apparatus of claim 2, wherein the dielectric layer comprises discontinuous portions, each portion disposed on the skin-facing surface of each electrode element of the at least one electrode element.

8. The apparatus of claim 2, wherein the electrode subassembly further comprises a circuit board, wherein the at least one electrode element is coupled to the circuit board, wherein the dielectric layer and the circuit board cooperate to encapsulate the at least one electrode element.

9. The apparatus of claim 2, wherein the electrode subassembly further comprises an adhesive layer or an adhesive structure positioned between the at least one electrode element and the dielectric layer.

10. The apparatus of claim 9, wherein the at least one polymer of the dielectric layer is or comprises a silicone rubber or elastomer, and wherein the adhesive layer or adhesive structure comprises a conductive silicone adhesive.

11. The apparatus of claim 2, wherein the at least one polymer of the dielectric layer is or comprises a silicone rubber or elastomer, and wherein the electrode subassembly further comprises a conductive layer comprising a silicone polymer.

12. The apparatus of claim 2, wherein the skin facing surface of the electrode subassembly is smooth and not tacky, and wherein the skin contact subassembly contacts the skin facing surface of the electrode subassembly.

13. The apparatus of claim 2, wherein the skin contact subassembly further comprises a layer of anisotropic conductive material.

14. The apparatus of claim 13, wherein the layer of anisotropic conductive material has a skin-facing side with a skin-facing surface and an opposing outwardly facing surface, wherein the layer of anisotropic conductive material is disposed in contact with the skin contact conductive adhesive or gel, and wherein the at least one electrode element is in electrical contact with the outwardly facing surface of the layer of anisotropic conductive material when the electrode subassembly is in contact with the skin contact subassembly.

15. The apparatus of claim 13, wherein the skin contact subassembly comprises a three layer unit comprising the skin contact conductive adhesive or gel, the layer of anisotropic conductive material, and a second conductive adhesive or gel layer; wherein the layer of anisotropic conductive material has a skin facing side with a skin facing surface and an opposing outwardly facing surface; and wherein the skin facing surface of the layer of anisotropic conductive material is in contact with the skin contact conductive adhesive or gel, and the outwardly facing surface of the layer of anisotropic conductive material is in contact with the second conductive adhesive or gel layer.

16. The apparatus of claim 13, wherein the layer of anisotropic conductive material is or comprises graphite.

17. The apparatus of claim 2, wherein the electrode subassembly further comprises a layer of anisotropic conductive material.

18. A method of using the apparatus of claim 2, the method comprising:
removing the skin contact subassembly from the electrode subassembly.

19. The method of claim 18, further comprising positioning a new skin contact subassembly against the skin-facing surface of the electrode subassembly so that the new skin contact subassembly is removably coupled to the electrode subassembly.

20. An apparatus comprising:
an electrode subassembly comprising:
at least one electrode element having a skin-facing side and a skin-facing surface; and
a dielectric layer on the skin-facing side of the at least one electrode element, wherein the dielectric layer has a dielectric constant of at least 10 and comprises a silicone rubber or elastomer having particles dispersed therethrough that are configured to provide an increased dielectric constant of the dielectric layer,
wherein the electrode subassembly comprises a skin-facing surface and wherein the dielectric layer provides the skin-facing surface of the electrode subassembly; and
a skin contact subassembly coupled to the electrode subassembly, wherein the skin contact subassembly comprises a skin contact conductive adhesive or gel configured to contact skin of a subject, wherein the skin contact conductive adhesive or gel is electrically coupled to the at least one electrode element when the skin contact subassembly is disposed against the skin-facing surface of the electrode subassembly; and
wherein the skin contact subassembly is releasably coupled to the electrode subassembly.
